(19) United States
(12) Patent Application Publication
(10) Pub. No.: US 2025/0076301 A1
(43) Pub. Date: Mar. 6, 2025

(54) BIOMARKER FOR PROSTATE CANCER

(71) Applicant: National Taiwan University, Taipei (TW)

(72) Inventors: Yeong-Shiau PU, Taipei (TW); Chung-Hsin CHEN, Taipei (TW); Pei-Wen HSIAO, Taipei (TW); Ming-Shyue LEE, Taipei (TW); Hsiang-Po HUANG, Taipei (TW); Kai-Hsiung CHANG, Taipei (TW)

(73) Assignee: National Taiwan University, Taipei (TW)

(21) Appl. No.: 18/825,767

(22) Filed: Sep. 5, 2024

Related U.S. Application Data
(60) Provisional application No. 63/580,581, filed on Sep. 5, 2023.

(51) Int. Cl. G01N 33/574

(52) U.S. Cl. CPC G01N 33/57484; G01N 33/57434; G01N 2800/342; G01N 2800/50

(57) ABSTRACT

Provided is a method of accurate and sensitive characterization and prognosis of prostate cancer in a subject. The method includes obtaining a biological sample from the subject and determining the level of identified biomarkers.

### BACKGROUND

### 1. Technical Field

This disclosure relates to methods for monitoring prostate cancer in a subject in need thereof. This disclosure also relates to methods and kits for detecting, diagnosing, prognosing, and characterizing prostate cancer in a subject in need thereof.

### 2. Description of Associated Art

Prostate cancer is the commonest non-epithelial cancer in men in the developed countries. Approximately 9 million new cases are diagnosed worldwide annually, and approximately 260,000 deaths occur due to prostate cancer. If prostate cancer is discovered early, 90% of the cases may be cured with surgery with the five-year survival rate for localized cancer at 100%. However, upon progression, the survival rate drops to less than 50%. It is, therefore, important to diagnose the cancer as early as possible and to monitor closely and effectively.

As of present, prostate cancer is screened using digital rectal examination (DRE), an imaging test such as transrectal ultrasound (TRUS), MRI, or a “fusion” of the two, and/or the measurement of the serum levels of prostate specific antigen (PSA). However, these approaches have low sensitivity and specificity due to high false-positives. For example, more than half of people screened with an elevated PSA level actually do not have prostate cancer as determined by subsequent confirmatory prostate biopsies. This implies that invasive biopsies are done more than needed. Indeed, many complications such as infection, internal bleeding, allergic reactions, impotence, and urinary incontinence can be resulted from invasive needle biopsies. These unnecessary biopsies and the accompanying complications lead to increased cost to the already burdened healthcare system. Obviously, there is an unmet need for safe and efficient prostate cancer screening and tumor grading system to improve the accuracy of prostate cancer detection and further risk stratification.

In addition to an accurate diagnosis, an effective cancer treatment regimen involves many different considerations and strategies. Following the diagnosis of cancer, an informative and accurate characterization of a cancer stage is of crucial importance in determining the proper treatment regimen, along with consideration of different aspects of patient, such as age and other disease history. It is valuable to determine an appropriate treatment for patient along the progression of the disease, and to ensure that precious clinical resources are targeted as effectively as possible on those that will benefit most from primary treatment (surgery, radiotherapy or active surveillance) and may also benefit from the most intensive post-treatment follow-up, and additional treatment upon recurrence where necessary (e.g., anti-androgens, androgen synthesis inhibitors, chemotherapy, beamline radiotherapy). As such, the current tests are not specific and robust enough to screen for prostate cancer. More reliable biological markers for providing prostate cancer diagnosis, risk stratification and prognoses and for monitoring disease progression are in need.

### SUMMARY

Herein, the present disclosure is therefore provided with groups of biomarkers and a method to characterize, diagnose, prognosticate, stratify and monitor the progression or recurrence of prostate cancer in a subject in need thereof. By the method of the present disclosure, the subject for characterization, diagnosis, monitoring and determining prognosis of cancer is able to receive a personalized treatment plan and/or customized healthcare, and accordingly an improved life quality is ensured when compared to ordinary methods prior to this disclosure.

The present disclosure provides a method to characterize prostate cancer in a subject in need thereof, comprising detecting a level of a prostate cancer marker in a biological sample from the subject, wherein the prostate cancer marker comprises one or more of the metabolite markers in Tables 1, 2, 5 and 6.

In one embodiment of the present disclosure, the prostate cancer marker comprises at least one metabolite marker selected from the group consisting of Ethanimidic acid, N-(trimethylsilyl)-, trimethylsilyl ester; ethanolamine; Glycine, di-TMS; pyruvic acid; Beta-alanine 1; L-(+) lactic acid; 2-hydroxypyridine; Diethanolamine, 3TMS derivative; glyceric acid; Pentenoic acid, 4-[(trimethylsilyl)oxy]-, trimethylsilyl ester; guanidinoacetic acid 2; tartronic acid; Butanoic acid, 2,4-bis[(trimethylsilyl) oxy]-, trimethylsilyl ester; L-pyroglutamic acid; DL-isoleucine 2; 1H-Indole, 1-(trimethylsilyl)-5-[(trimethylsilyl)oxy]; 2,3,4-Trihydroxybutyric acid tetrakis(trimethylsilyl) deriv., (, (R*,R*)—); 1-Deoxypentitol, 4TMS derivative; 4-hydroxybenzoic acid; 4-acetamidobutyric acid 1; L-glutamine 2; D-lyxose 2; Arabinofuranose, 1,2,3,5-tetrakis-O-(trimethylsilyl); xanthine; Ribitol TMS; xylitol; L-(−)-Arabitol, 5TMS derivative; Furan, tetrahydro-2,5-dipropyl-; 1,5-anhydro-D-sorbitol; L-Phenylalanine, 2TMS derivative; 3,4-Dihydroxyphenylacetic Acid, 3TMS derivative; DL-4-hydroxymandelic acid; 3-methyl-L-histidine; trans-aconitic acid; Ethyl (E)-1-penten-3-ynesulfonate; D-allose 2; D-allose 1; L-tyrosine 2; quinic acid; galacturonic acid 2; Ononitol TMS; D-Gluconic acid, 6TMS derivative; pantothenic acid 2; N-acetyl-D-mannosamine 1; D-Allose, pentakis(trimethylsilyl) ether, ethyloxime (isomer 2); Pseudo uridine penta-tms; palmitic acid; 2-phenyl-3,5,7-tris(trimethylsilyloxy)-1-benzopyran-4-one; stearic acid; Guanosine, N,N-dimethyl-1-(trimethylsilyl)-2′,3′,5′-tris-O-(trimethylsilyl)-; 1-Monopalmitin, 2TMS derivative; lactose 1; 2-Monostearin, 2TMS derivative; 1-stearoyl-rac-glycerol; 3-Phenyl-5,10-secocholesta-1(10),2-dien-5-one.

In one embodiment of the present disclosure, the biological sample is peripheral blood, sera, plasma, urine, semen, prostatic fluid, Cowper's fluid, or pre-ejaculatory fluid and any combination thereof.

In one embodiment of the present disclosure, the method further comprising detecting a level of prostate specific antigen in the biological sample from the subject.

In one embodiment of the present disclosure, the method further comprising grouping the subject by NCCN risk classification into six groups of different severities of prostate cancer, wherein the six groups are benign group, very low-risk/low-risk prostate cancer, favorable-intermediate-risk prostate cancer, unfavorable-intermediate-risk prostate cancer, high-risk/very high-risk prostate cancer, and metastasis prostate cancer group.

In one embodiment of the present disclosure, the method further comprising distinguishing the severity of prostate cancer in the subject in one group from the other groups.

The present disclosure further provides a method for determining a need of biopsy for prostate cancer diagnosis in a subject in need thereof, comprising detecting a level of a prostate cancer marker in a biological sample from the subject, wherein the prostate cancer marker is selected from the group consisting of panels in Tables 3, 4, 7 and 8.

In one embodiment of the present disclosure, the prostate cancer marker is selected from the group consisting of panel 1, panel 2, panel 3, panel 4, and any combination thereof, and wherein:
panel 1 is selected from the group consisting of C₁₀ H₂₁ N₄O₂, C₁₂H₁₇NO, C₁₂H₂NOPS, C₁₂H₉O₉P, C₁₃H₁₉N₅O₅, C₁₇H₃₂N₃O₇, C₁₈H₁₆N₆O₃, C₁₈H₃₃NO₄, C₁₈H₄₃N₄O₃, C₁₉H₃₅NO₅, C₁₉H₃₈N₂O₃, C₂₄H₄₂N₇O₃, C₂₇H₁₂N₉, C₃₄H₂₃N₇O₅, C₅H₁₁NO, C₅₁H₂₉N₅O₄, C₆H₁₅N, C₈H₉N, C₉H₄N₅O₉, C₉H₈O₂, and any combination thereof;
panel 2 is selected from the group consisting of C₁₁H₅NOPS, C₁₂H₁₆NO₇, C₁₂H₂ NOPS, C₁₂H₉O₉P, C₁₃H₂₅NO₂, C₁₃H₂₅NO₃, C₁₄H₃₀N₄O₂, C₁₆H₁₃N₃O₃P, C₁₇H₄₁N₄O₃, C₁₉H₁₉N₈, C₂₂H₄₅NO₄, C₂₆H₅₁N₄O₅, C₂₆H₅₈N₁₃P, C₂₇H₁₂N₉, C₂₇H₅₅N₈O₃, C₃₀H₅₇NO₇, C₃₀H₆₄N₁₅O₂P, C₄₁H₂₃N₁₁O₂, C₅, C₅H₇NO₃, C₆HCl₅, C₆H₁₆N₃O₅, C₈H₁₆NO₅, and any combination thereof;
panel 3 is selected from the group consisting of C₁₀H₁₈N₂O₅, C₁₂H₂₁NO₄, C₁₃H₂₃NO₆, C₁₃H₂₅NO₃, C₁₄H₃₀N₄O₂, C₁₅H₃₀N₁₀OP, C₁₆H₁₃N₃O₃P, C₁₉H₃₁N₆O₂, C₂₂H₄₅NO₄, C₂₇H₁₂N₉, C₂₈H₅₇N₈O₄, C₃₀H₆₁N₈O₅, C₃₀H₆₄N₁₅O₂P, C₃₅H₇₁N₈O₇, C₄₀H₃₈N₂₂O₄, C₄₁H₂₃N₁₁O₂, C₄₃H₄₀N₂₀O₃, C₅H₁₁NO, C₅H₁NO₂S, C₅H₂O₂P, C₈H₁₆NO₅, and any combination thereof; and
panel 4 is selected from the group consisting of C₁₁H₂₀O₂, C₁₁H₅NOPS, C₁₂H₂NOPS, C₁₂H₂₅NO₄P, C₁₂H₉O₉P, C₁₃H₂₅NO₂, C₁₃H₂₅NO₃, C₁₄H₃₀N₄O₂, C₁₆H₃₀N₃O₂, C₁₇H₄₁N₄O₃, C₁₈H₃₄O₅, C₁₉H₃₁N₆O₂, C₂₁H₃₆N₄O₃, C₂₂H₄₅NO₄, C₂₃H₄₇N₈O₂, C₂₄H₄₁N₁₄O₈, C₂₇H₁₂N₉, C₃₀H₅₇NO₇, C₃₀H₆₄N₁₅O₂P, C₃₅H₇₁N₈O₇, C₄₃H₄₀N₂₀O₃, C₅H₁₁NO, C₅H₁NO₂S, C₆H₁₄N₂O₅P, and any combination thereof.

In one embodiment of the present disclosure, the prostate cancer marker is selected from the group consisting of panel 5, panel 6, panel 7, panel 8, and any combination thereof, and wherein:
panel 5 is selected from the group consisting of C₁₀H₁₆O₄, C₁₀H₁₈N₂O₄, C₁₁H₂₀NO₃P₃, C₁₂H₇N₄O₂, C₁₅H₂₈N₆OP₂, C₁₆H₃₉N₈OP, C₁₉H₁₄O₃, C₂₁H₃₃N₃O₃, C₂₃H₂₇O₁₁S, C₂₃H₄₂N₇O, C₂₅H₄₆N₇O₃, C₂₇H₄₈P₂, C₂₇H₅₄O₆, C₃₃H₂₂O₇, C₃₄H₇₃N₈O₂P, C₃₈H₄₈O₁₂, C₄₀H₈₅NOP₃, C₅H₄O₃, C₅H₈N₂O₂, C₆H₁₃N₄O₃P, C₆H₁₃N₄OP₂, C7H₁₀O₄, C7H₁₇O₇P₂, C7H₆O₆S, C₈H₁₄O₄, and any combination thereof;
panel 6 is selected from the group consisting of C₁₀H₁₆O₄, C₁₁H₁₆N₄O₄, C₁₂H₇N₄O₂, C₁₄H₂₀N₂O₅, C₁₆H₃₂O₂, C₁₇H₃₄N₉O₂, C₂₁H₃₃N₃O₃, C₂₃H₂₇O₁₁S, C₂₆H₄₃NO₆, C₂₇H₄₈P₂, C₂₈H₅₂N₇O, C₃₄H₇₃N₈O₂P, C₃₈H₄₈O₁₂, C₃₉H₂₆O₇, C₄H₆O₄, C₄₀H₈₅NOP₃, C₅H₄N₄O₂, C₅H₈N₂O₂, C₆H₁₁N₄O₂P, C₆H₁₃N₄OP₂, C₆H₆N₄O₂, C₆H₈N₂O₄, C7H₁₀O₄, C7H₁₇O₇P₂, C7H₆O₆S, C₉H₁₆O₄, C₉H₉NO₃, and any combination thereof;
panel 7 is selected from the group consisting of C₁₀H₁₈N₂O₄, C₁₀H₁₉N₅P₃, C₁₂H₇N₄O₂, C₁₅H₂₈N₆OP₂, C₁₆H₃₂O₂, C₁₇H₃₄N₉O₂, C₁₉H₁₄O₃, C₂₁H₃₃N₃O₃, C₂₁H₃₉N₄OP, C₂₇H₄₈P₂, C₃₈H₄₈O₁₂, C₃₉H₂₆O₇, C₄₀H₈₅NOP₃, C₅H₁₀N₂O₃, C₅H₄N₄O₂, C₆H₁₀O₄S, C₆H₁₁N₄O₂P, C₆H₁₃N₄OP₂, C₆H₁₅O₈P, C7H₁₀O₄, C7H₁₇O₇P₂, C7H₂₁N₃OP₃, C7H₂₂N₄O₉PS, C7H₈O₆S, C₈H₉O₆, C₉H₁₆O₄, C₉H₁₇N O₄S, C₉H₉NO₃, and any combination thereof; and
panel 8 is selected from the group consisting of C₁₀H₁₉N₅P₃, C₁₂H₇N₄O₂, C₁₅H₂₈N₆OP₂, C₁₇H₃₄N₉O₂, C₁₇H₄₂N₃OP₂, C₂₁H₃₃N₃O₃, C₂₂H₃₈N₇O, C₂₄H₄₀N₄O₃, C₂₅H₄₆N₇O₃, C₂₅ H₅₀O₆, C₂₇H₅₄O₆, C₃₉H₂₆O₇, C₃₉H₇₈O₆, C₄₀H₈₅NOP₃, C₆H₁₁N₄O₂P, C₆H₁₅O₈P, C₆H₅N₂OP, C₆H₈O₆S, C7H₁₀O₄, C7H₁₇O₇P₂, C7H₂₁N₃OP₃, C₈H₁₆N₂O₅P, C₈H₁₈NO₆P, C₈H₄N₄O₃, C₉H₁₆O₄, C₉H₉NO₃, and any combination thereof.

In one embodiment of the present disclosure, the prostate cancer marker is selected from the group consisting of panel 9, panel 10, panel 11, panel 12, and any combination thereof, and wherein:
panel 9 is selected from the group consisting of Pyruvic acid, 4-Acetamidobutyric acid, 1,5-Anhydro-D-glucitol, Beta-Alanine, Glyceric acid, D-Lyxose, Galacturonic acid, D-Allose, L-Tyrosine, 3-Methyl-L-histidine, L-Glutamine, L-Pyroglutamic acid, Guanidinoacetic acid, Lactose, 2-Hydroxypyridine, N-acetyl-D-mannosamine, Palmitic acid, 1-Deoxy-d-ribitol, Monopalmitin, 2-Stearoylglycerol, Galangin, 6-ethoxyiminohexane-1,2,3,4,5-pentol, D-Gluconic acid, N,N-Dimethylguanosine, Pseudouridine, Ribitol, and any combination thereof;
panel 10 is selected from the group consisting of Pyruvic acid, Xanthine, 4-Acetamidobutyric acid, 1,5-Anhydro-D-glucitol, Beta-Alanine, 1-Stearoyl-rac-glycerol, Glyceric acid, Galacturonic acid, Quinic acid, Xylitol, L-Pyroglutamic acid, Guanidinoacetic acid, Lactose, Ononitol, 5-Hydroxyindole, Monopalmitin, Galangin, 3,4-Dihydroxyphenylacetic acid, 3-Phenyl-5,10-secocholesta-1 (10),2-dien-5-one, Acetamide, Ethyl 1-penten-3-ynesulfonate, 2,5-Dipropyltetrahydrofuran, L-Phenylalanine, Pseudouridine, and any combination thereof;
panel 11 is selected from the group consisting of L-Lactic acid, Xanthine, 4-Acetamidobutyric acid, Beta-Alanine, 1-Stearoyl-rac-glycerol, 4-hydroxymandelic acid, trans-Aconitic acid, D-Allose, Tartronic acid, Stearic acid, L-Tyrosine, Quinic acid, Ethanolamine, Guanidinoacetic acid, DL-isoleucine, Palmitic acid, Monopalmitin, Arabinofuranose, 2,4-Dihydroxybutanoic acid, Diethanolamine, Acetamide, 2,5-Dipropyltetrahydrofuran, Glycine, L-Arabinitol, Levulinic acid, Pseudouridine, and any combination thereof; and
panel 12 is selected from the group consisting of Pyruvic acid, Xanthine, 4-Hydroxybenzoic acid, Beta-Alanine, 1-Stearoyl-rac-glycerol, Galacturonic acid, D-Allose, Tartronic acid, Quinic acid, Pantothenic acid, Xylitol, Guanidinoacetic acid, 1-Deoxy-d-ribitol, Monopalmitin, Threonic acid, Galangin, Arabinofuranose, 2,4-Dihydroxybutanoic acid, D-Gluconic acid, 2,5-Dipropyltetrahydrofuran, Levulinic acid, Pseudouridine, and any combination thereof.

In one embodiment of the present disclosure, the prostate cancer marker is selected from the group consisting of panel 13, panel 14, panel 15, panel 16, and any combination thereof, and wherein:
panel 13 is selected from the group consisting of 1-Methoxymethyl-2-phenylthioindole-3-carbaldehyde, 2,3-Dihydroxybutanoic acid, 2-Hydroxypyridine, 2-Stearoylglycerol, 3-Hydroxyphenylacetic acid, 3-Indoleacetic acid, 3-Methyl-L-histidine, 4-Acetamidobutyric acid, 4-hydroxymandelic acid, 6-ethoxyiminohexane-1,2,3,4,5-pentol, alpha-Hydroxyisobutyric acid, D-Altrose, D-Gluconic acid, D-Lyxose, Galacturonic acid, Galangin, Glyceric acid, Lactose, L-Fucose, L-Pyroglutamic acid, Monopalmitin, Ononitol, Oxamide, Palmitic acid, Pseudouridine, Pyruvic acid, Ribitol, trans-Aconitic acid and any combination thereof;
panel 14 is selected from the group consisting of 1-Stearoyl-rac-glycerol, 2,5-Dipropyltetrahydrofuran, 3,4-Dihydroxyphenylacetic acid, Acetamide, Beta-Alanine, Cyclohexylamine, Ethyl 1-penten-3-ynesulfonate, Galacturonic acid
Galangin, Glyceric acid, Guanidinoacetic acid, Levulinic acid, Monopalmitin, Ononitol, Palmitic acid, p-Tolyl-beta-D-glucopyranosid-uronsaeure, Quinic acid, Stearic acid, Sucrose, Uric acid, Xanthine, Xylitol, and any combination thereof;
panel 15 is selected from the group consisting of (22S,23S,25R)-3β-methoxy-16β,23:22,26-diepoxy-5α-cholestane, 1-Methoxymethyl-2-phenylthioindole-3-carbaldehyde, 1-Stearoyl-rac-glycerol, 2,4-Dihydroxybutanoic acid, 2,5-Dipropyltetrahydrofuran, 4-hydroxymandelic acid, Acetamide, Arabinofuranose, Beta-Alanine, Daidzein, D-Allose, DL-isoleucine, D-tagatofuranose, Ethanolamine, Galangin, Guanidinoacetic acid, L-Arabinitol, Levulinic acid, L-Lactic acid, Monopalmitin, Palmitic acid, Pseudouridine, Quinic acid, Stearic acid, Sucrose, Tartronic acid, Xanthine, and any combination thereof; and
panel 16 is selected from the group consisting of (4RS,5SR)-5-hydroperoxy-4-decanol, 2,5-Dipropyltetrahydrofuran, 3,4,5-Trihydroxypentanoic acid, 4-Hydroxybenzoic acid, 6-ethoxyiminohexane-1,2,3,4,5-pentol, Acetamide, Arabinofuranose, Beta-Alanine, D-Allose, DL-4-Hydroxy-3-methoxymandelic acid, Ethyl 1-penten-3-ynesulfonate, Galacturonic acid, Galangin, Glyceric acid, Guanidinoacetic acid, Hippuric Acid, Levulinic acid, L-Pyroglutamic acid, Pantothenic acid, Pseudouridine, Pyruvic acid, Quinic acid, Tartronic acid, Uric acid, Xanthine, and any combination thereof.

The present disclosure further provides a method for monitoring a prostate cancer subject on active surveillance (AS), comprising detecting a level of a prostate cancer marker in a biological sample from the subject, wherein the prostate cancer marker is selected from the group consisting of markers in Tables 1, 2, 5 and 6.

### DETAILED DESCRIPTION OF THE PREFERRED EMBODIMENTS

The present disclosure provides a method and biomarkers to diagnose, stratify, prognosticate and monitor prostate cancer in a subject in need thereof by analyzing the levels of one or more biomarker in a sample obtained from the subject. All terms including descriptive or technical terms which are used herein should be construed as having meanings that are obvious to one of ordinary skill in the art. However, the terms may have different meanings according to an intention of one of ordinary skill in the art, case precedents, or appearance of new technologies. Also, some terms may be arbitrarily selected by the applicant, and in this case, the meaning of the selected terms will be described in detail in the comprehensive descriptions of the present disclosure. Thus, the terms used herein have to be defined based on meaning of the terms together with descriptions throughout the specification.

Also, when a part “includes” or “comprises” a component or a step, unless there is a particular description contrary thereto, the part can further include other components or other steps, not excluding the others.

It is further noted that, as used in this disclosure, the singular forms “a,” “an,” and “the” include plural referents unless expressly and unequivocally limited to one referent. The term “or” is used interchangeably with the term “and/or” unless the context clearly indicates otherwise.

The term “to characterize” in a subject or individual may include, but is not limited to, to provide the diagnosis of a disease or a condition, to determine the stratification of a disease risk, to assess the risk of a disease, to provide the prognosis of a disease or a condition, to determine a disease stage or a condition stage, to determine the severity of a disease, to evaluate the malignancy potential of a disease, to monitor a recurrence of cancer, to evaluate a drug efficacy, to describe a physiological condition, to evaluate an organ distress or organ rejection, to monitor disease or condition progression, to determine therapy-related association to a disease or a condition, or to describe a specific physiological or biological state.

As used herein, prognosis of cancer may include predicting the clinical outcome of the patient, assessing the risk of cancer recurrence, determining treatment modality, or determining treatment efficacy.

As used herein, the term “metastasis” describes the spread of a cancer from one part of the body to another. A tumor formed by cells that have spread can be called a “metastatic tumor” or a “metastasis.” The metastatic tumor often contains cells that are similar to those in the original (primary) tumor, and have, but not limited to, genomic, epigenetic, transcriptomic, and metabolic alterations.

As used herein, the term “progression” describes the course of a disease, such as a cancer, as it becomes worse or spreads in the body.

The terms “subject,” “patient” and “individual” are used interchangeably herein and refer to a warm-blooded animal, such as a mammal that is afflicted with, or suspected of having, at risk for or being pre-disposed to, or being screened for cancer, e.g., actual or suspected cancer. These terms include, but are not limited to, domestic animals, sports animals, primates and humans. For example, the terms refer to a human.

The term “detect,” “detecting” or “detection” includes assaying, or otherwise establishing the presence or absence of the target biomarker(s), subunits, or combinations of reagent-bound targets, and the like, or assaying for ascertaining, establishing, characterizing, predicting or otherwise determining one or more factual characteristics of a cancer such as stage, aggressiveness, metastatic potential or patient survival, or assisting with the same. A cut-off value or a standard may correspond to levels quantitated for samples from control healthy subjects with no disease or low-grade cancer or from other samples of the subject.

As used herein, the term “marker” or “biomarker” is a biological molecule, or a panel of biological molecules, whose altered level in a tissue, cell or sample as compared to its level in normal or healthy tissue, cell or sample is associated with a disease state, such as an abnormal prostate state, including disease in an early stage, e.g., prior to the detection of one or more symptoms associated with the disease. In an aspect of the disclosure, prostate cancer may be characterized by identifying and measuring the level of one or more biomarkers listed in Tables 1 to 8 in a biological sample.

The biological sample obtained from the subject may be any bodily fluid. For example, the biological sample can be peripheral blood, sera, plasma, ascites, urine, cerebrospinal fluid (CSF), sputum, saliva, bone marrow, synovial fluid, aqueous humor, cerumen, bronchoalveolar lavage fluid, semen, prostatic fluid, Cowper's fluid or pre-ejaculatory fluid, sweat, fecal matter, hair, tears, cyst fluid, pleural and peritoneal fluid, pericardial fluid, lymph, chyme, chyle, bile, interstitial fluid, pus, sebum, vomit, mucosal secretion, stool water, pancreatic juice, lavage fluids from sinus cavities, bronchopulmonary aspirates or other lavage fluids.

In one embodiment, the marker is detected in a urine sample. In another embodiment, the marker is detected in a blood sample, e.g., serum or plasma. In one embodiment, the marker is detected in serum. In one embodiment, the marker is detected in plasma. In some embodiments, the serum or plasma can be further processed to remove abundant blood proteins (e.g., albumin) or irrelevant proteins that are not marker proteins prior to analysis.

Examples of biomarkers include, but are not limited to, polypeptides, peptides, polypeptide fragments, antibodies, hormones, polynucleotides, RNA or RNA fragments, microRNAs (miRNAs), lipids, metabolites, or polysaccharides. In some embodiments, biomarker may be a metabolite marker. In one embodiment, the severity of prostate cancer in a subject can be determined or predicted by a panel of biomarkers or by a combination of two panels that are established by metabolites markers, respectively, through the processes including, but not limited to, K-fold cross validation, forward selection, reverse selection, logistic regression, and/or decision tree analysis. As such, the disclosure can effectively improve the predication index such as, but not limited to, area under the curve (AUC), sensitivity, specificity, positive predictive value (PPV), or negative predictive value (NPV). In some embodiment, the efficacy of combination of two panels of metabolite markers may be better than that of individual panel thereof. As used herein, “panel” refers to a particular combination of biomarkers that is used to determine or predict severity of prostate cancer in a subject, and assign subjects into different groups according to the severity of prostate cancer. As used herein, “group” refers to a selection of subjects being determined to have similar condition or severity of prostate cancer. As used herein, a “model” refers to use of different panels of biomarkers in determining and assigning potential prostate cancer patients in different groups of severity.

In some embodiments, the biomarker involves in the pathway such as, but not limited to, fatty acid biosynthesis, purine metabolism, tryptophan metabolism, pyrimidine metabolism, arginine proline metabolism, pentose and glucuronate interconversion, valine degradation/pyrimidine metabolism, glyoxylate metabolism, ubiquinone biosynthesis, or any combination thereof. In one embodiment, the biomarker is a metabolite marker.

### EXAMPLE

Exemplary embodiments of the present disclosure are further described in the following examples, which do not limit the scope of the present disclosure.

### Example 1: Grouping of Prostate Cancer Patients

For efficient identification of prostate cancer patients with different severities, Gleason's pattern scale (from grade 1 to 5) is assigned to each prostate tissue biopsy core by experienced pathologist. Grade 1 is given to cells that look like normal prostate tissue while the grade 5 is assigned to cancer cells with very abnormal growth patterns. Most prostate cancers score a grade of 3 or higher. Grade 1 and 2 are not used in the biopsy reports. Prostate tumors are often made up of multiple foci with different grades. Two grades are usually assigned for each patient to give rise to a Gleason sum or Gleason scores. A primary grade is given to describe the cells that make up the largest area of the tumor, and a second grade is given to describe cells of the next largest area. Based on patient's disease risk and severities, six different groups were used to stratify patients in a more precise manner.

The first group is a benign group where no cancer was found; the second group is the metastatic prostate cancer group (mPC) with cancer cells breaking the prostate capsule barrier and invading into other organs (e.g., lymph nodes or bones); the third group is very-low-risk/low-risk prostate cancer group (VLR/LR PC) with all below criteria: Gleason score less than or equal to 6 (e.g., 3+3, the first 3 is the primary grade and the second 3 is the secondary grade), clinical T1 to T2a stage, and PSA of 10 ng/mL or less; the fourth group is high-risk/very-high-risk prostate cancer group (HR/VHR PC) with one of the below criteria: clinical T3a or more, Gleason sum of 8 or more, and PSA of more than 20 ng/mL; the fifth and sixth groups are intermediate-risk prostate cancer group with at least one intermediate-risk criteria below: clinical T2b-2c, Gleason score of 4+3 or 3+4, and PSA of 10 to 20 ng/mL. Among them, the fifth group is favorable-intermediate-risk prostate cancer group (FIR PC) with the below three criteria: only one intermediate-risk factor, Gleason score of 3+4 or less, and less than 50% biopsy cores positive for prostate cancer. The sixth group is the unfavorable-intermediate-risk prostate cancer group (UIR PC) with one of the below three criteria: 2 or 3 intermediate-risk factors, Gleason score of 4+3, and 50% or more of biopsy cores positive for prostate cancer.

Currently, assessing and dividing potential prostate cancer patients into these six groups as mentioned above rely on invasive needle biopsy. With the present disclosure herewith, efficient assessment of patients can be made with the use of corresponding panel of biomarkers with a proper method of analysis. Different models, through the use of different panels of biomarkers, to determine and distinguish potential prostate cancer patients in different groups of severity is adopted and useful under various clinical scenarios. These models identify and distinguish a potential prostate cancer patient in one or more severity groups from the rest of the groups. For example, patients can be distinguished between the benign group versus the rest groups, VLR/LR PC, FIR PC, UIR PC, HR/VHR PC and mPC, for population screening or general health checkup. In another population screening or health check-up, VLR/LR PC can be regarded as benign and divided the subjects under test into a group of benign and VLR/LR PC versus another group consisting of FIR PC, UIR PC, HR/VHR PC and mPC. In another clinical scenario with elder prostate cancer patients, such as those older than 75 years old, an analysis to distinguish between the group of benign, VLR/LR PC or FIR PC versus the group of UIR PC, HR/VHR PC and mPC would be meaningful, considering the risk derived from VLR/LR PC or FIR PC may unlikely blunt a life-span expectation of a man older than 75 years old.

While in a clinical scenario involving a new positive biopsy that the patient is in need of risk stratification and prognosis, an analysis dividing the subject between the group of VLR/LR PC or FIR PC versus the group of UIR PC, HR/VHR PC and mPC is required. Another clinical scenario that could find this analysis useful is for monitoring prostate cancer among patients with VLR/LR PC or FIR PC under active surveillance (AS).

Furthermore, when there is a young prostate cancer patient with a new positive biopsy, then an analysis on whether he belongs to VLR/LR PC versus FIR/UIR/HR/VHR PC or mPC group is useful, considering that the risk of FIR/UIR/HR/VHR PC or mPC may significantly blunt his life-span expectation and impair his social-economical contribution, if not diagnosed in time and properly treated. This analysis that distinguishes an VLR/LR PC group from FIR/UIR/HR/VHR PC or mPC groups is also meaningful for a young prostate cancer patient seeking for AS options.

Therefore, for monitoring patients under AS, different models of comparison and/or panels of markers can be used based on the age of the patient, other physiological condition or clinical manifestations, e.g., PSA level. Doctors can decide which model of comparison and/or panels of markers to be used to allow the best AS option for each patient. For example, for elder patients such as those aged greater than 75-year-old, the AS will adopt the model of comparison that distinguishes benign, VLR/LR PC or FIR PC from those of UIR/HR/VHR PC or mPC, and for younger patients such as those aged less than 60-year-old, the AS will adopt the model of comparison that distinguishes benign, VLR/LR PC from those of FIR/UIR/HR/VHR PC or mPC.

### Example 2: Identification of Metabolite Markers for assessing prostate cancer Risk and Assigning Patients in Different Group of Severity Using Liquid Chromatography-Mass Spectrometry (LC/MS) Analysis

Two modes of metabolite analysis were carried out with different columns using liquid chromatography-mass spectrometry (LC/MS) analysis, which are the positive mode with BEH C18 column and negative mode with HILIC column. For positive mode, the urine samples were diluted with water (1:10 vol/vol), and then centrifuged at 4° C. and 13200 rpm for 10 minutes. The supernatants were then transferred to the new sample vial for LC/MS analysis with respective columns.

The LC/MS system used is Agilent 1290 Infinity II ultra-performance liquid chromatography (UPLC) system (Agilent Technologies, Palo Alto, CA, USA) coupled online to the Dual AJS electrospray ionization (ESI) source of an Agilent 6545 quadrupole time-of-flight (Q-TOF) mass spectrometer (Agilent Technologies, Palo Alto, CA, USA). The sample was separated by using ACQUITY UPLC BEH C18 column (1.7 μm, 2.1×100 mm, Waters Corp., Milford, MA, USA) and ACQUITY UPLC BEH amide column (1.7 μm, 2.1×100 mm, Waters Corp., Milford, MA, USA). The column temperature was 40° C. The mobile phase for BEH C18 column was H₂O (eluent A) and acetonitrile (eluent B), both eluents with 0.1% formic acid. The gradient condition was: 0 to 1 min, 2% B; 1 to 4 min, 2 to 40% B; 4 to 8 min, 40 to 70% B; 8 to 10 min, 70 to 95% B; 10 to 12 min, 95% B; 12 to 13 min, 95 to 2% B; 13 to 16 min, 2% B. The flow rate was 400 μL/min, and the injection volume of sample was 1 μL. The mobile phase for BEH amide column was H₂O (eluent A) and 90% acetonitrile (eluent B), both eluents with 15 mM ammonium acetate and 0.3% NH₄OH. The gradient condition was: 0 to 7 min, 90% B; 7 to 8 min, 70 to 50% B; 8 to 10 min, 50% B; 10 to 11 min, 50 to 90% B; 11 to 16 min, 90% B. The post time of elution was 4 min. The flow rate was 300 μL/min, and the injection volume of sample was 2 μL. The instrument was operated in positive full-scan mode with BEH C18 column and negative full-scan mode with BEH amide column, both methods collected from an m/z of 60 to 1700. The MS operating conditions were optimized as follows: Vcap voltage, 3.5 kV; nozzle voltage, 0.5 kV; nebulizer, 45 psi; gas temperature, 300° C.; sheath gas temperature, 325° C.; sheath gas flow (nitrogen), 8 L/min; drying gas (nitrogen), 8 L/min.

For negative mode with HILIC column, the urine samples were diluted with acetonitrile (1:10 vol/vol), then centrifuged at 4° C. and 13200 rpm for 10 min. The supernatants were transferred to the new sample vial for LC/MS analysis. The LC/MS method is same with BEH C18 column as described above.

The chromatogram acquisition, detection of mass spectral peaks, and their waveform processing were performed using Agilent Qualitative Analysis 10.0 and Agilent Profinder 10.0 software (Agilent, USA).

To identify and select the specific metabolites as markers for distinguishing different groups, a univariate logistic regression to select differentially accumulated metabolites with P values less than 0.1. The identified differential compounds were further analyzed by Receiver operating characteristic (ROC), using Medcalc software version 11.2 (Medcalc Software, Belgium). Furthermore, a K-fold cross validation and a followed reverse selection-based logistic regression were applied to select discriminator sets with improved AUC performance.

To find urine biomarkers that distinguish prostate tumors with different malignancy potential, four different models of comparison were performed by analyzing metabolites from LC/MS with BEH C18 column. The union of all these four sets of markers from LC/MS with BEH C18 column is listed in Table 1 below.

TABLE 1
Mass to charge ratio (m/z) of the union of all sets of potential
metabolite markers from LC/MS with BEH C18 column for distinguishing
patients of prostate tumor-with different malignancy potential

| Marker | | Retention | | CAS |
|---|---|---|---|---|
| metabolites | m/z | time (min) | Annotation by SIRIUS | Number |
| C₅ | 59.9999 | 0.6 | — | — |
| C₅H₁₁NO | 101.0842 | 13.2 | N-Butylformamide | 871-71-6 |
| C₆H₁₅N | 101.1201 | 13.2 | — | — |
| C₈H₉N | 119.0732 | 1.9 | Isoindoline | 496-12-8 |
| C₅H₂O₂P | 124.9791 | 0.6 | — | — |
| C₅H₇NO₃ | 129.0427 | 3.2 | 5-Oxo-D-proline | 4042-36-8 |
| C9H₈O₂ | 148.052 | 7.4 | Pyruvophenone | 579-07-07 |
| C₅H₁₁NO₂S | 149.0505 | 0.9 | Methionine | 59-51-8 |
| C₁₁H₂₀O₂ | 184.1458 | 5.4 | Undecylenic acid | 112-38-9 |
| C₁₂H₁₇NO | 191.1302 | 5.6 | Hexanilide | 621-15-8 |
| C₈H₁₆NO₅ | 206.1025 | 2.5 | — | — |
| C₆H₁₆N₃O₅ | 210.1089 | 0.7 | — | — |
| C₆H₁₄N₂O₅P | 225.0635 | 2.7 | Vanilloylglycine | 1212-04-0 |
| C₁₃H₂₅NO₂ | 227.1883 | 5.7 | Undecylenamide MEA | 20545-92-0 |
| C₁₀H₂₁N₄O₂ | 229.1673 | 5 | N-decanoylglycine | 14305-32-9 |
| C₁₁H₅NOPS | 229.9828 | 0.9 | — | — |
| C₁₂H₂NOPS | 238.9582 | 0.6 | — | — |
| C₁₂H₂₁NO₄ | 243.1467 | 3 | 3-(Cyclobutanecarbonyloxy)-4- | — |
| | | | (trimethylazaniumyl)butanoate | |
| C₁₃H₂₅NO₃ | 243.1826 | 5.1 | N-Undecanoylglycine | 83871-09-4 |
| C₁₀H₁₈N₂O₅ | 246.1209 | 2.7 | — | — |
| C₆HCl₅ | 247.8566 | 13.4 | — | — |
| C₁₂H₂₅NO₄P | 278.1509 | 9 | Dibutyl phthalate | 84-74-2 |
| C₁₂ H₁₆NO₇ | 286.0931 | 3.2 | — | — |
| C₁₄H₃₀N₄O₂ | 286.2362 | 0.7 | — | — |
| C₁₃H₂₃NO₆ | 289.1519 | 2.4 | O-adipoyl-L-carnitine | 102636-83-9 |
| C₁₆H₃₀N₃O₂ | 296.2339 | 8.1 | 10-Hydroxyoctadeca-12,15- | 34932-14-4 |
| | | | dienoic acid | |
| C₁₃H₁₉N₅O₅ | 325.1379 | 2 | N(2),N(2),7-trimethylguanosine | — |
| C₉H₄N₅O₉ | 325.9998 | 6.6 | — | — |
| C₁₆H₁₃N₃O₃P | 326.0699 | 8.1 | — | — |
| C₁₈H₃₃NO₄ | 327.2396 | 7 | — | — |
| C₁₂H₉O₉P | 327.9969 | 6.6 | — | — |
| C₁₈H₃₄O₅ | 330.2395 | 5.7 | — | — |
| C₁₉H₃₈N₂O₃ | 342.2868 | 6.3 | Cocamidopropyl betaine | 4292/10/8 |
| C₁₇H₄₁N₄O₃ | 349.3176 | 10.3 | — | — |
| C₁₉H₃₅NO₅ | 357.2518 | 6.3 | [3-carboxy-2-[(Z)-3- | — |
| | | | hydroxydodec-9- | |
| | | | enoyl]oxypropyl]- | |
| | | | trimethylazanium | |
| C₁₉H₁₉N₈ | 359.1742 | 8.5 | N-(1,4-Dihydroxy-4- | 2058332-33-3 |
| | | | methylpentan-2-YL)-3-hydroxy- | |
| | | | 5-oxo-6-phenylhexanamide | |
| C₁₈H₄₃N₄O₃ | 363.3334 | 10.8 | 1,2-Propanediol, 3-((2- | 34719-62-5 |
| | | | hydroxyheptadecyl)oxy)- | |
| C₁₈H₁₆N₆O₃ | 364.1293 | 8.6 | — | — |
| C₁₉H₃₁N₆O₂ | 375.2519 | 6.6 | 1,3,5-Tris(2,2- | 745070-61-5 |
| | | | dimethylpropionylamino)benzene | |
| C₂₂H₄₅NO₄ | 387.3335 | 11.6 | — | — |
| C₁₇H₃₂N₃O₇ | 390.2231 | 2.5 | — | — |
| C₂₁H₃₆N₄O₃ | 392.2789 | 6.6 | (2R)-N-[(2S)-1-amino-3- | 1212507-31-7 |
| | | | cyclohexyl-1-oxopropan-2-yl]-1- | |
| | | | (cyclohexanecarbonyl)piperazine- | |
| | | | 2-carboxamide | |
| C₁₅H₃₀N₁₀OP | 397.2335 | 6.6 | Lysylthreonyllysine | 106326-71-0 |
| C₂₇H₁₂N₉ | 462.1229 | 10 | — | — |
| C₂₃H₄₇N₈O₂ | 467.3828 | 9.8 | — | — |
| C₂₄H₄₂N₇O₃ | 476.3347 | 6.6 | — | — |
| C₂₆H₅₁N₄O₅ | 499.388 | 10.2 | — | — |
| C₂₇H₅₅N₈O₃ | 539.4401 | 10.7 | — | — |
| C₃₀H₅₇NO₇ | 543.4126 | 10.2 | — | — |
| C₂₈H₅₇N₈O₄ | 569.4509 | 10.2 | — | — |
| C₂₆H₅₈N₁₃P | 583.4642 | 10.7 | — | — |
| C₃₄H₂₃N₇O₅ | 609.1765 | 12.1 | — | — |
| C₃₀H₆₁N₈O₅ | 613.477 | 10.2 | — | — |
| C₂₄H₄₁N₁₄O₈ | 653.3222 | 10.4 | — | — |
| C₃₀H₆₄N₁₅O₂P | 697.5124 | 10.7 | — | — |
| C₄₁H₂₃N₁₁O₂ | 701.2021 | 12 | — | — |
| C₃₅H₇₁N₈O₇ | 715.5414 | 10.6 | — | — |
| C₅₁H₂₉N₅O₄ | 775.2244 | 12.9 | — | — |
| C₄₃H₄₀N₂₀O₃ | 884.3598 | 11.2 | — | — |
| C₄₀H₃₈N₂₂O₄ | 890.3456 | 12.4 | — | — |

To find urine biomarkers that distinguish prostate tumors with different malignancy potential, metabolites from LC/MS with HILIC column in four different models of comparison were analyzed. The union of all these four sets of markers from LC/MS with HILIC column for distinguishing different malignancy potential of prostate tumors is listed in Table 2 below.

TABLE 2
Mass to charge ratio (m/z) of the union of all sets of potential
metabolite markers from LC/MS with HILIC column for distinguishing
prostate tumors with different malignancy potential

| | | Retention | | |
|---|---|---|---|---|
| Marker | | time | Annotation by | CAS |
| metabolites | m/z | (min) | SIRIUS | Number |
| C₅H₄O₃ | 112.016 | 5.1 | 2-Furoate | 88-14-2 |
| C₄H₆O₄ | 118.0263 | 4 | Succinate | 110-15-6 |
| C₅H₈N₂O₂ | 128.0578 | 1.6 | 1,3-Diazepane- | 75548-99-1 |
| | | | 2,4-dione | |
| C₅H₁₀N₂O₃ | 146.0691 | 1.6 | Alanylglycine | 1188-01-8 |
| C₆H₅N₂OP | 152.0134 | 1.8 | — | — |
| C₅H₄N₄O₂ | 152.0328 | 2.4 | Xanthine | 69-89-6 |
| C₇H₁₀O₄ | 158.0575 | 3.7 | Hept-2- | 1085697- |
| | | | enedioic acid | 38-6 |
| C₆H₆N₄O₂ | 166.0491 | 1.6 | 1-Methylxanthine | 6136-37-4 |
| C₆H₈N₂O₄ | 172.0481 | 2.6 | Hydantoin- | 5624-26-0 |
| | | | propionate | |
| C₈H₁₄O₄ | 174.089 | 3.4 | Suberic acid | 505-48-6 |
| C₆H₁₀O₄S | 178.03 | 1.5 | 3,3′- | — |
| | | | Thiodipropanoate | |
| C₉H₉NO₃ | 179.0584 | 1.4 | Hippurate | 495-69-2 |
| C₉H₁₆O₄ | 188.1045 | 3.2 | Azelaic acid | 123-99-9 |
| C₁₀H₁₆O₄ | 200.1049 | 1 | Radioplex | 10018-78-7 |
| C₈H₉O₆ | 201.0396 | 1.4 | — | — |
| C₆H₁₁N₄O₂P | 202.0602 | 2.3 | — | — |
| C₈H₄N₄O₃ | 204.0299 | 1.6 | — | — |
| C₆H₈O₆S | 208.0033 | 1 | — | — |
| C7H₆O₆S | 217.9884 | 1.6 | Salicylsulfuric | 89-45-2 |
| | | | acid | |
| C₆H₁₁N₄O₃P | 218.055 | 3.4 | — | — |
| C₆H₁₃N₄OP₂ | 219.0564 | 1.9 | S-(3-Oxopropyl)- | 140226-30-8 |
| | | | N-acetylcysteine | |
| C₇H₈O₆S | 220.0041 | 0.9 | 1-Methyl- | — |
| | | | pyrogallol-3-O- | |
| | | | sulphate | |
| C₁₀H₁₈N₂O₄ | 230.1258 | 2 | DI-Acetyl-lysine | 499-86-5 |
| C₉H₁₇NO₄S | 235.0868 | 1.6 | S-(D- | 1632078- |
| | | | Carboxybutyl)-L- | 43-3 |
| | | | homocysteine | |
| C₁₂H₇N₄O₂ | 239.0556 | 1.9 | — | — |
| C₆H₁₅O₈P | 246.0504 | 3.4 | — | — |
| C₈H₁₆N₂O₅P | 251.0787 | 1.5 | N- | 1220-05-9 |
| | | | Feruloylglycine | |
| C₈H₁₈NO₆P | 255.0875 | 2.1 | Pantothenate | 79-83-4 |
| C7H₂₁N₃OP₃ | 256.0895 | 4.5 | — | — |
| C₁₆H₃₂O₂ | 256.2403 | 0.9 | Hexadecanoic | 1957/10/3 |
| | | | acid | |
| C₁₁H₁₆N₄O₄ | 268.1169 | 3.1 | Acetylcarnosine | — |
| C7H₁₇O₇P₂ | 275.0449 | 1 | L-Tyrosine | 81660-41-5 |
| | | | methyl ester 4- | |
| | | | sulfate | |
| C₁₉H₁₄O₃ | 290.0942 | 0.8 | — | — |
| C₁₄H₂₀N₂O₅ | 296.1362 | 1.5 | — | — |
| C₁₀H₁₉N₅P₃ | 302.0856 | 1.6 | — | — |
| C₁₁H₂₀NO₃P₃ | 307.0652 | 2.7 | — | — |
| C₇H₂₂N₄O₉PS | 369.084 | 2.8 | — | — |
| C₁₅H₂₈N₆OP₂ | 370.18 | 0.9 | DHT-sulfate | 2641-48-7 |
| C₂₁H₃₃N₃O₃ | 375.2509 | 0.9 | — | — |
| C₁₆H₃₉N₈OP | 390.2988 | 0.9 | — | — |
| C₂₁H₃₉N₄OP | 394.2848 | 1.7 | — | — |
| C₁₇H₄₂N₅OP₂ | 394.2849 | 0.9 | — | — |
| C₁₇H₃₄N₉O₂ | 396.2825 | 1.7 | — | — |
| C₂₂H₃₈N₇O | 416.3138 | 1 | — | — |
| C₂₄H₄₀N₄O₃ | 432.3084 | 0.9 | — | — |
| C₂₃H₄₂N₇O | 432.3413 | 0.9 | — | — |
| C₂₇H₄₈P₂ | 434.3237 | 0.9 | — | — |
| C₂₅H₅₀O₆ | 446.3605 | 0.9 | — | — |
| C₂₆H₄₃NO₆ | 465.3085 | 1.7 | — | — |
| C₂₇H₅₄O₆ | 474.3916 | 0.9 | — | — |
| C₂₅H₄₆N₇O₃ | 492.3639 | 0.9 | — | — |
| C₂₈H₅₂N₇O | 502.4229 | 0.9 | — | — |
| C₂₃H₂₇O₁₁S | 511.1262 | 1.4 | — | — |
| C₃₃H₂₂O₇ | 530.1361 | 0.8 | — | — |
| C₃₉H₂₆O₇ | 606.1673 | 0.8 | — | — |
| C₃₉H₇₈O₆ | 642.5768 | 0.9 | — | — |
| C₃₄H₇₃N₈O₂P | 656.5583 | 0.9 | — | — |
| C₄₀H₈₅NOP₃ | 688.5843 | 1 | — | — |
| C₃₈H₄₈O₁₂ | 696.3159 | 0.9 | — | — |

In each of different models of comparison designed for diverse clinical scenarios, a representative panel of metabolite markers was identified from LC/MS with BEH C18 column, as shown in Table 3 below, with their prediction ability evaluated by AUC analysis. Sensitivity and specificity of the prediction are also shown in bracket following AUC.

TABLE 3
Representative panels of metabolite markers from LC/MS with BEH C18 column for
distinguishing patients of prostate tumors with different malignancy potential

| | | | AUC | AUC |
|---|---|---|---|---|
| | | | (sensitivity/ | (sensitivity/ |
| | Models of | Metabolite markers | specificity) | specificity) |
| No | comparison | (N = Number of markers) | without PSA | with PSA |
| 1 | Benign | N = 20 | 0.89 (90%/55%) | 0.91 (90%/64%) |
| | vs. | C₁₀H₂₁N₄O₂ | | |
| | VLR PC/LR | C₁₂H₁₇NO | | |
| | PC/FIR PC/UIR | C₁₂H₂NOPS | | |
| | PC/HR PC/VHR | C₁₂H₉O₉P | | |
| | PC/mPC | C₁₃H₁₉N₅O₅ | | |
| | | C₁₇H₃₂N₃O₇ | | |
| | | C₁₈H₁₆N₆O₃ | | |
| | | C₁₈H₃₃NO₄ | | |
| | | C₁₈H₄₃N₄O₃ | | |
| | | C₁₉H₃₅NO₅ | | |
| | | C₁₉H₃₈N₂O₃ | | |
| | | C₂₄H₄₂N₇O₃ | | |
| | | C₂₇H₁₂N₉ | | |
| | | C₃₄H₂₃N₇O₅ | | |
| | | C₅H₁₁NO | | |
| | | C₅₁H₂₉N₅O₄ | | |
| | | C₆H₁₅N | | |
| | | C₈H₉N | | |
| | | C₉H₄N₅O₉ | | |
| | | C₉H₈O₂ | | |
| 2 | Benign/VLR | N = 23 | 0.84 (90%/49%) | 0.89 (90%/62%) |
| | PC/LR PC | C₁₁H₅NOPS | | |
| | vs. | C₁₂H₁₆NO₇ | | |
| | FIR PC/UIR | C₁₂H₂NOPS | | |
| | PC/HR PC/VHR | C₁₂H₉O₉P | | |
| | PC/mPC | C₁₃H₂₅NO₂ | | |
| | | C₁₃H₂₅NO₃ | | |
| | | C₁₄H₃₀N₄O₂ | | |
| | | C₁₆H₁₃N₃O₃P | | |
| | | C₁₇H₄₁N₄O₃ | | |
| | | C₁₉H₁₉N₈ | | |
| | | C₂₂H₄₅NO₄ | | |
| | | C₂₆H₅₁N₄O₅ | | |
| | | C₂₆H₅₈N₁₃P | | |
| | | C₂₇H₁₂N₉ | | |
| | | C₂₇H₅₅N₈O₃ | | |
| | | C₃₀H₅₇NO₇ | | |
| | | C₃₀H₆₄N₁₅O₂P | | |
| | | C₄₁H₂₃N₁₁O₂ | | |
| | | C₅ | | |
| | | C₅H₇NO₃ | | |
| | | C₆HCl₅ | | |
| | | C₆H₁₆N₃O₅ | | |
| | | C₈H₁₆NO₅ | | |
| 3 | Benign/VLR | N = 21 | 0.78 (90%/47%) | 0.86 (90%/61%) |
| | PC/LR PC/FIR PC | C₁₀H₁₈N₂O₅ | | |
| | vs. | C₁₂H₂₁NO₄ | | |
| | UIR PC/HR | C₁₃H₂₃NO₆ | | |
| | PC/VHR PC/mPC | C₁₃H₂₅NO₃ | | |
| | | C₁₄H₃₀N₄O₂ | | |
| | | C₁₅H₃₀N₁₀OP | | |
| | | C₁₆H₁₃N₃O₃P | | |
| | | C₁₉H₃₁N₆O₂ | | |
| | | C₂₂H₄₅NO₄ | | |
| | | C₂₇H₁₂N₉ | | |
| | | C₂₈H₅₇N₈O₄ | | |
| | | C₃₀H₆₁N₈O₅ | | |
| | | C₃₀H₆₄N₁₅O₂P | | |
| | | C₃₅H₇₁N₈O₇ | | |
| | | C₄₀H₃₈N₂₂O₄ | | |
| | | C₄₁H₂₃N₁₁O₂ | | |
| | | C₄₃H₄₀N₂₀O₃ | | |
| | | C₅H₁₁NO | | |
| | | C₅H₁₁NO₂S | | |
| | | C₅H₂O₂P | | |
| | | C₈H₁₆NO₅ | | |
| 4 | Benign/PC with | N = 24 | 0.82 (90%/48%) | 0.86 (90%/57%) |
| | GS < 7 | C₁₁H₂₀O₂ | | |
| | vs. | C₁₁H₅NOPS | | |
| | PG with GS >= 7 | C₁₂H₂NOPS | | |
| | | C₁₂H₂₅NO₄P | | |
| | | C₁₂H₉O₉P | | |
| | | C₁₃H₂₅NO₂ | | |
| | | C₁₃H₂₅NO₃ | | |
| | | C₁₄H₃₀N₄O₂ | | |
| | | C₁₆H₃₀N₃O₂ | | |
| | | C₁₇H₄₁N₄O₃ | | |
| | | C₁₈H₃₄O₅ | | |
| | | C₁₉H₃₁N₆O₂ | | |
| | | C₂₁H₃₆N₄O₃ | | |
| | | C₂₂H₄₅NO₄ | | |
| | | C₂₃H₄₇N₈O₂ | | |
| | | C₂₄H₄₁N₁₄O₈ | | |
| | | C₂₇H₁₂N₉ | | |
| | | C₃₀H₅₇NO₇ | | |
| | | C₃₀H₆₄N₁₅O₂P | | |
| | | C₃₅H₇₁N₈O₇ | | |
| | | C₄₃H₄₀N₂₀O₃ | | |
| | | C₅H₁₁NO | | |
| | | C₅H₁₁NO₂S | | |
| | | C₆H₁₄N₂O₅P | | |

In each of different models of comparison designed for diverse clinical scenarios, a representative panel of metabolite markers from LC/MS with HILIC column was also identified, as shown in Table 4 below. The prediction ability was evaluated by AUC analysis, with or without inclusion of PSA level in calculation. Sensitivity and specificity of the prediction is shown in bracket following AUC.

TABLE 4
Representative panels of metabolite markers from LC/MS with HILIC column
for distinguishing prostate tumors with different malignancy potential

| | | | AUC | AUC |
|---|---|---|---|---|
| | | | (sensitivity/ | (sensitivity/ |
| | Models of | Metabolite markers | specificity) | specificity) |
| No | comparison | (N = Number of markers) | without PSA | with PSA |
| 1 | Benign | N = 25 | 0.86 (90%/51%) | 0.88 (90%/65%) |
| | vs. | C₁₀H₁₆O₄ | | |
| | VLR PC/LR | C₁₀H₁₈N₂O₄ | | |
| | PC/FIR PC/UIR | C₁₁H₂₀NO₃P₃ | | |
| | PC/HR PC/VHR | C₁₂H₇N₄O₂ | | |
| | PC/mPC | C₁₅H₂₈N₆OP₂ | | |
| | | C₁₆H₃₉N₈OP | | |
| | | C₁₉H₁₄O₃ | | |
| | | C₂₁H₃₃N₃O₃ | | |
| | | C₂₃H₂₇O₁₁S | | |
| | | C₂₃H₄₂N₇O | | |
| | | C₂₅H₄₆N₇O₃ | | |
| | | C₂₇H₄₈P₂ | | |
| | | C₂₇H₅₄O₆ | | |
| | | C₃₃H₂₂O₇ | | |
| | | C₃₄H₇₃N₈O₂P | | |
| | | C₃₈H₄₈O₁₂ | | |
| | | C₄₀H₈₅NOP₃ | | |
| | | C₅H₄O₃ | | |
| | | C₅H₈N₂O₂ | | |
| | | C₆H₁₁N₄O₃P | | |
| | | C₆H₁₃N₄OP₂ | | |
| | | C7H₁₀O₄ | | |
| | | C7H₁₇O₇P₂ | | |
| | | C7H₆O₆S | | |
| | | C₈H₁₄O₄ | | |
| 2 | Benign/VLR | N = 27 | 0.80 (90%/46%) | 0.86 (90%/53%) |
| | PC/LR PC | C₁₀H₁₆O₄ | | |
| | vs. | C₁₁H₁₆N₄O₄ | | |
| | FIR PC/UIR | C₁₂H₇N₄O₂ | | |
| | PC/HR PC/VHR | C₁₄H₂₀N₂O₅ | | |
| | PC/mPC | C₁₆H₃₂O₂ | | |
| | | C₁₇H₃₄N₉O₂ | | |
| | | C₂₁H₃₃N₃O₃ | | |
| | | C₂₃H₂₇O₁₁S | | |
| | | C₂₆H₄₃NO₆ | | |
| | | C₂₇H₄₈P₂ | | |
| | | C₂₈H₅₂N₇O | | |
| | | C₃₄H₇₃N₈O₂P | | |
| | | C₃₈H₄₈O₁₂ | | |
| | | C₃₉H₂₆O₇ | | |
| | | C₄H₆O₄ | | |
| | | C₄₀H₈₅NOP₃ | | |
| | | C₅H₄N₄O₂ | | |
| | | C₅H₈N₂O₂ | | |
| | | C₆H₁₁N₄O₂P | | |
| | | C₆H₁₃N₄OP₂ | | |
| | | C₆H₆N₄O₂ | | |
| | | C₆H₈N₂O₄ | | |
| | | C7H₁₀O₄ | | |
| | | C7H₁₇O₇P₂ | | |
| | | C7H₆O₆S | | |
| | | C₉H₁₆O₄ | | |
| | | C₉H₉NO₃ | | |
| 3 | Benign/VLR | N = 28 | 0.77 (90%/42%) | 0.86 (90%/56%) |
| | PC/LR PC/FIR PC | C₁₀H₁₈N₂O₄ | | |
| | vs. | C₁₀H₁₉N₅P₃ | | |
| | UIR PC/HR | C₁₂H₇N₄O₂ | | |
| | PC/VHR PC/mPC | C₁₅H₂₈N₆OP₂ | | |
| | | C₁₆H₃₂O₂ | | |
| | | C₁₇H₃₄N₉O₂ | | |
| | | C₁₉H₁₄O₃ | | |
| | | C₂₁H₃₃N₃O₃ | | |
| | | C₂₁H₃₉N₄OP | | |
| | | C₂₇H₄₈P₂ | | |
| | | C₃₈H₄₈O₁₂ | | |
| | | C₃₉H₂₆O₇ | | |
| | | C₄₀H₈₅NOP₃ | | |
| | | C₅H₁₀N₂O₃ | | |
| | | C₅H₄N₄O₂ | | |
| | | C₆H₁₀O₄S | | |
| | | C₆H₁₁N₄O₂P | | |
| | | C₆H₁₃N₄OP₂ | | |
| | | C₆H₁₅O₈P | | |
| | | C7H₁₀O₄ | | |
| | | C7H₁₇O₇P₂ | | |
| | | C7H₂₁N₃OP₃ | | |
| | | C7H₂₂N₄O₉PS | | |
| | | C7H₈O₆S | | |
| | | C₈H₉O₆ | | |
| | | C₉H₁₆O₄ | | |
| | | C₉H₁₇NO₄S | | |
| | | C₉H₉NO₃ | | |
| 4 | Benign/PC with | N = 26 | 0.81 (90%/53%) | 0.87 (90%/57%) |
| | GS < 7 | C₁₀H₁₉N₅P₃ | | |
| | vs. | C₁₂H₇N₄O₂ | | |
| | PG with GS >= 7 | C₁₅H₂₈N₆OP₂ | | |
| | | C₁₇H₃₄N₉O₂ | | |
| | | C₁₇H₄₂N₅OP₂ | | |
| | | C₂₁H₃₃N₃O₃ | | |
| | | C₂₂H₃₈N₇O | | |
| | | C₂₄H₄₀N₄O₃ | | |
| | | C₂₅H₄₆N₇O₃ | | |
| | | C₂₅H₅₀O₆ | | |
| | | C₂₇H₅₄O₆ | | |
| | | C₃₉H₂₆O₇ | | |
| | | C₃₉H₇₈O₆ | | |
| | | C₄₀H₈₅NOP₃ | | |
| | | C₆H₁₁N₄O₂P | | |
| | | C₆H₁₅O₈P | | |
| | | C₆H₅N₂OP | | |
| | | C₆H₈O₆S | | |
| | | C7H₁₀O₄ | | |
| | | C7H₁₇O₇P₂ | | |
| | | C7H₂₁N₃OP₃ | | |
| | | C₈H₁₆N₂O₅P | | |
| | | C₈H₁₈NO₆P | | |
| | | C₈H₄N₄O₃ | | |
| | | C₉H₁₆O₄ | | |
| | | C₉H₉NO₃ | | |

### Example 3: Identification of Metabolite Markers for Assessing Prostate Cancer Risk and Grouping of Patients Using Gas Chromatography-Mass Spectrometry (GC/MS) Analysis

First, urine sample preparation started with incubating an individual urine sample with urease enzyme to deplete excess urea, as a high abundance of urea is a major chromatographic interference. 100 U of urease was added to 100 μL of each human urine sample, followed by incubation at 37° C. with mild shaking at 650 rpm for 1 hour to decompose and remove excess urea. Subsequently, termination of urease activity and extraction of metabolites were carried out by admixing 1 mL of methanol with vortex for 30 seconds, and precipitated proteins were removed by centrifugation at 13,200 rpm for 15 min at 4° C. The supernatants were transferred to a 2-mL microcentrifugation tube and then dried in SpeedVac vacuum concentrators. The dried metabolic extract was derivatized by bis(trimethylsilyl)-trifluoroacetamide (BSTFA) containing 1% trimethylchlorosilane (TMCS) and analyzed using GC/MS as explained below.

The derivatized samples were analyzed using Agilent 7890B gas chromatography coupled with 7250 quadrupole time-of-flight mass spectrometer (GC-Q-TOF/MS) equipped with electron ionization (EI). The separation was performed on Zorbax DB5-MS+10 m Duragard Capillary Column (30 m×0.25 mm×0.25 mm, Agilent). The GC temperature profile was held at 60° C. for 1 minute and then raised at 10° C./min to 325° C. and held at 325° C. for 10 minutes. The transfer line and the ion source temperature were set at 300° C. and 280° C., respectively. The mass range monitored was from 50 to 600 Daltons. Mass spectra were compared against the NIST 2017, Fiehn, and Wiley Registry 11th Edition mass spectral library.

A univariate logistic regression to select differentially accumulated metabolites with P values less than 0.1. The identified differential compounds were further analyzed by Receiver operating characteristic (ROC), using Medcalc software version 11.2 (Medcalc Software, Belgium). Furthermore, a K-fold cross validation and a followed reverse selection-based logistic regression were applied to select discriminator sets with improved AUC performance.

To identify urine biomarkers that distinguish prostate tumors with different malignancy potential, five different models of comparison analyzing metabolites from GC/MS were performed. The union of all these five sets of markers is listed in Table 5. For the patients with PSA less than 20 ng/ml, the union of these five sets of markers is shown in Table 6.

TABLE 5
The union of all sets of potential metabolite markers from GC/MS for
distinguishing prostate tumors with different malignancy potential

| Metabolite markers | CAS number | MW g/mol |
|---|---|---|
| Ethanimidic acid, N-(trimethylsilyl)-, trimethylsilyl ester | 60-35-5 | 59.07 |
| [700] ethanolamine [9.879] | 141-43-5 | 61.08 |
| Glycine, di-TMS | 56-40-6 | 75.07 |
| [1060] pyruvic acid [6.714] | 127-17-3 | 88.06 |
| [239] Beta-alanine 1 [12.044] | 107-95-9 | 89.09 |
| [107689] L-(+) lactic acid [6.851] | 79-33-4 | 90.08 |
| [8871] 2-hydroxypyridine [6.519] | 142-08-05 | 95.1 |
| Diethanolamine, 3TMS derivative | 111-42-2 | 105.14 |
| [439194] glyceric acid [10.735] | 473-81-4 | 106.08 |
| Pentenoic acid, 4-[(trimethylsilyl)oxy]-, trimethylsilyl ester | 123-76-2 | 116.12 |
| [763] guanidinoacetic acid 2 [14.751] | 352-97-6 | 117.108 |
| [45] tartronic acid [11.523] | 80-69-3 | 120.06 |
| Butanoic acid, 2,4-bis[(trimethylsilyl)oxy]-, trimethylsilyl ester | 1518-62-3 | 120.1 |
| [7405] L-pyroglutamic acid [13.218] | 98-79-3 | 129.11 |
| [791] DL-isoleucine 2 [10.225] | 443-79-8 | 131.17 |
| 1H-Indole, 1-(trimethylsilyl)-5-[(trimethylsilyl)oxy]- | 1953-54-4 | 133.15 |
| 2,3,4-Trihydroxybutyric acid tetrakis(trimethylsilyl) deriv., (, (R*,R*)-) | 3909/12/4 | 136.1 |
| 1-Deoxypentitol, 4TMS derivative | 13046-76-9 | 136.15 |
| [135] 4-hydroxybenzoic acid [14.505] | 99-96-7 | 138.12 |
| [18189] 4-acetamidobutyric acid 1 [12.863] | 3025-96-5 | 145.16 |
| [738] L-glutamine 2 [14.083] | 56-85-9 | 146.14 |
| [439240] D-lyxose 2 [14.889] | 1114-34-7 | 150.13 |
| Arabinofuranose, 1,2,3,5-tetrakis-O-(trimethylsilyl)- | 13221-22-2 | 150.13 |
| [1188] xanthine [18.574] | 69-89-6 | 152.11 |
| Ribitol TMS | 488-81-3 | 152.15 |
| [6912] xylitol [15.376] | 87-99-0 | 152.15 |
| L-(−)-Arabitol, 5TMS derivative | 7643-75-6 | 152.15 |
| Furan, tetrahydro-2,5-dipropyl- | 4457-62-9 | 156.26 |
| [219984] 1,5-anhydro-D-sorbitol [16.967] | 154-58-5 | 164.16 |
| L-Phenylalanine, 2TMS derivative | 63-91-2 | 165.19 |
| 3,4-Dihydroxyphenylacetic Acid, 3TMS derivative | 102-32-9 | 168.15 |
| [328] DL-4-hydroxymandelic acid [16.126] | 1198-84-1 | 168.15 |
| [64969] 3-methyl-L-histidine [16.423] | 368-16-1 | 169.18 |
| [444212] trans-aconitic acid [15.842] | 4023-65-8 | 174.11 |
| Ethyl (E)-1-penten-3-ynesulfonate | 171816-65-2 | 174.22 |
| [448388] D-allose 2 [17.521] | 2595-97-3 | 180.156 |
| [448388] D-allose 1 [17.278] | 2595-97-3 | 180.16 |
| [6057] L-tyrosine 2 [17.856] | 60-18-4 | 181.19 |
| [6508] quinic acid [17.076] | 77-95-2 | 192.17 |
| [445929] galacturonic acid 2 [18.105] | 685-73-4 | 194.14 |
| Ononitol TMS | 6090-97-7 | 194.18 |
| D-Gluconic acid, 6TMS derivative | 526-95-4 | 196.16 |
| [6613] pantothenic acid 2 [18.371] | 79-83-4 | 219.23 |
| [899] N-acetyl-D-mannosamine 1 [19.177] | 7772-94-3 | 221.21 |
| D-Allose, pentakis(trimethylsilyl) ether, ethyloxime (isomer 2) | 2058302-87-5 | 223.22 |
| Pseudo uridine penta-tms | 1445-07-04 | 244.2 |
| [985] palmitic acid [18.846] | 1957/10/3 | 256.4 |
| 2-phenyl-3,5,7-tris(trimethylsilyloxy)-1-benzopyran-4-one | 548-83-4 | 270.24 |
| [5281] stearic acid [20.675] | 1957/11/4 | 284.48 |
| Guanosine, N,N-dimethyl-1-(trimethylsilyl)-2′,3′,5′-tris-O-(trimethylsilyl)- | 2140-67-2 | 311.29 |
| 1-Monopalmitin, 2TMS derivative | 542-44-9 | 330.5 |
| [84571] lactose 1 [24.386] | 63-42-3 | 342.3 |
| 2-Monostearin, 2TMS derivative | 621-61-4 | 358.56 |
| [24699] 1-stearoyl-rac-glycerol [24.913] | 123-94-4 | 358.6 |
| 3-Phenyl-5,10-secocholesta-1(10),2-dien-5-one | —* | 460.74 |

TABLE 6
The union of all sets of potential metabolite markers from
GC/MS for distinguishing prostate tumors (with PSA level
less than 20 ng/mL) with different malignancy potential

| No. | Name of metabolites |
|---|---|
| 1 | (4RS,5SR)-5-hydroperoxy-4-decanol |
| 2 | (22S,23S,25R)-3β-methoxy-16β,23:22,26-diepoxy-5α-cholestane |
| 3 | 1-Methoxymethyl-2-phenylthioindole-3-carbaldehyde |
| 4 | 1-Stearoyl-rac-glycerol |
| 5 | 2,3-Dihydroxybutanoic acid |
| 6 | 2,4-Dihydroxybutanoic acid |
| 7 | 2,5-Dipropyltetrahydrofuran |
| 8 | 2-Hydroxypyridine |
| 9 | 2-Stearoylglycerol |
| 10 | 3,4,5-Trihydroxypentanoic acid |
| 11 | 3,4-Dihydroxyphenylacetic acid |
| 12 | 3-Hydroxyphenylacetic acid |
| 13 | 3-Indoleacetic acid |
| 14 | 3-Methyl-L-histidine |
| 15 | 4-Acetamidobutyric acid |
| 16 | 4-Hydroxybenzoic acid |
| 17 | 4-hydroxymandelic acid |
| 18 | 6-ethoxyiminohexane-1,2,3,4,5-pentol |
| 19 | Acetamide |
| 20 | alpha-Hydroxyisobutyric acid |
| 21 | Arabinofuranose |
| 22 | Beta-Alanine |
| 23 | Cyclohexylamine |
| 24 | Daidzein |
| 25 | D-Allose |
| 26 | D-Altrose |
| 27 | D-Gluconic acid |
| 28 | DL-4-Hydroxy-3-methoxymandelic acid |
| 29 | DL-isoleucine |
| 30 | D-Lyxose |
| 31 | D-tagatofuranose |
| 32 | Ethanolamine |
| 33 | Ethyl 1-penten-3-ynesulfonate |
| 34 | Galacturonic acid |
| 35 | Galangin |
| 36 | Glyceric acid |
| 37 | Guanidinoacetic acid |
| 38 | Hippuric Acid |
| 39 | Lactose |
| 40 | L-Arabinitol |
| 41 | Levulinic acid |
| 42 | L-Fucose |
| 43 | L-Lactic acid |
| 44 | L-Pyroglutamic acid |
| 45 | Monopalmitin |
| 46 | Ononitol |
| 47 | Oxamide |
| 48 | Palmitic acid |
| 49 | Pantothenic acid |
| 50 | Pseudouridine |
| 51 | p-Tolyl-beta-D-glucopyranosid-uronsaeure |
| 52 | Pyruvic acid |
| 53 | Quinic acid |
| 54 | Ribitol |
| 55 | Stearic acid |
| 56 | Sucrose |
| 57 | Tartronic acid |
| 58 | trans-Aconitic acid |
| 59 | Uric acid |
| 60 | Xanthine |
| 61 | Xylitol |

### Example 4: Metabolite Markers From GC/MS Analysis for Assessing Prostate Cancer Risk and Grouping of Patients

In each of different models of comparison designed for different patients, a representative panel of metabolite markers from GC/MS was identified, as shown in Table 7 below. The prediction ability was evaluated by AUC analysis, with or without inclusion of PSA level in calculation. Sensitivity and specificity of the prediction are also shown in bracket following AUC. For the patients with PSA less than 20 ng/ml, the representative panel of metabolite markers is listed in Table 8.

TABLE 7
Representative panels of GC/MS-derived metabolite markers for distinguishing
the malignancy potential of patients with prostate tumors

| | | | AUC | AUC |
|---|---|---|---|---|
| | | | (sensitivity/ | (sensitivity/ |
| | Models of | Metabolite markers | specificity) | specificity) |
| No | comparison | (N = Number of markers) | without PSA | with PSA |
| 1 | Benign | N = 26 | 0.94 | 0.94 |
| | vs. | Pyruvic acid | (90%/79%) | (90%/82%) |
| | VLR/LR/FIR/ | 4-Acetamidobutyric acid | | |
| | UIR/HR/VHR | 1,5-Anhydro-D-glucitol | | |
| | PC and mPC | Beta-Alanine | | |
| | | Glyceric acid | | |
| | | D-Lyxose | | |
| | | Galacturonic acid | | |
| | | D-Allose | | |
| | | L-Tyrosine | | |
| | | 3-Methyl-L-histidine | | |
| | | L-Glutamine | | |
| | | L-Pyroglutamic acid | | |
| | | Guanidinoacetic acid | | |
| | | Lactose | | |
| | | 2-Hydroxypyridine | | |
| | | N-acetyl-D-mannosamine | | |
| | | Palmitic acid | | |
| | | 1-Deoxy-d-ribitol | | |
| | | Monopalmitin | | |
| | | 2-Stearoylglycerol | | |
| | | Galangin | | |
| | | 6-ethoxyiminohexane- | | |
| | | 1,2,3,4,5-pentol | | |
| | | D-Gluconic acid | | |
| | | N,N-Dimethylguanosine | | |
| | | Pseudouridine | | |
| | | Ribitol | | |
| 2 | Benign and | N = 24 | 0.85 | 0.90 |
| | VLR/LR PC | Pyruvic acid | (90%/63%) | (90%/73%) |
| | vs. | Xanthine | | |
| | FIR/UIR/HR/ | 4-Acetamidobutyric acid | | |
| | VHR PC and | 1,5-Anhydro-D-glucitol | | |
| | mPC | Beta-Alanine | | |
| | | 1-Stearoyl-rac-glycerol | | |
| | | Glyceric acid | | |
| | | Galacturonic acid | | |
| | | Quinic acid | | |
| | | Xylitol | | |
| | | L-Pyroglutamic acid | | |
| | | Guanidinoacetic acid | | |
| | | Lactose | | |
| | | Ononitol | | |
| | | 5-Hydroxyindole | | |
| | | Monopalmitin | | |
| | | Galangin | | |
| | | 3,4-Dihydroxyphenylacetic | | |
| | | acid | | |
| | | 3-Phenyl-5,10-secocholesta- | | |
| | | 1(10),2-dien-5-one | | |
| | | Acetamide | | |
| | | Ethyl 1-penten-3-ynesulfonate | | |
| | | 2,5-Dipropyltetrahydrofuran | | |
| | | L-Phenylalanine | | |
| | | Pseudouridine | | |
| 3 | Benign and | N = 26 | 0.82 | 0.90 |
| | VLR/LR/FIR | L-Lactic acid | (90%/50%) | (90%/67%) |
| | PC | Xanthine | | |
| | vs. | 4-Acetamidobutyric acid | | |
| | UIR/HR/VHR | Beta-Alanine | | |
| | PC and mPC | 1-Stearoyl-rac-glycerol | | |
| | | 4-hydroxymandelic acid | | |
| | | trans-Aconitic acid | | |
| | | D-Allose | | |
| | | Tartronic acid | | |
| | | Stearic acid | | |
| | | L-Tyrosine | | |
| | | Quinic acid | | |
| | | Ethanolamine | | |
| | | Guanidinoacetic acid | | |
| | | DL-isoleucine | | |
| | | Palmitic acid | | |
| | | Monopalmitin | | |
| | | Arabinofuranose | | |
| | | 2,4-Dihydroxybutanoic acid | | |
| | | Diethanolamine | | |
| | | Acetamide | | |
| | | 2,5-Dipropyltetrahydrofuran | | |
| | | Glycine | | |
| | | L-Arabinitol | | |
| | | Levulinic acid | | |
| | | Pseudouridine | | |
| 4 | Benign + | N = 22 | 0.80 | 0.85 |
| | GS < 7 PC | Pyruvic acid | (90%/46%) | (90%/55%) |
| | vs. | Xanthine | | |
| | GS ≥ 7 PC | 4-Hydroxybenzoic acid | | |
| | | Beta-Alanine | | |
| | | 1-Stearoyl-rac-glycerol | | |
| | | Galacturonic acid | | |
| | | D-Allose | | |
| | | Tartronic acid | | |
| | | Quinic acid | | |
| | | Pantothenic acid | | |
| | | Xylitol | | |
| | | Guanidinoacetic acid | | |
| | | 1-Deoxy-d-ribitol | | |
| | | Monopalmitin | | |
| | | Threonic acid | | |
| | | Galangin | | |
| | | Arabinofuranose | | |
| | | 2,4-Dihydroxybutanoic acid | | |
| | | D-Gluconic acid | | |
| | | 2,5-Dipropyltetrahydrofuran | | |
| | | Levulinic acid | | |
| | | Pseudouridine | | |

TABLE 8
Representative panels of GC/MS metabolite markers for distinguishing the malignancy
potential of patients with prostate tumors (PSA level less than 20 ng/mL)

| | | | AUC | AUC |
|---|---|---|---|---|
| | | | (sensitivity/ | (sensitivity/ |
| | Models of | Metabolite markers | specificity) | specificity) |
| No | comparison | (N = Number of markers) | without PSA | with PSA |
| 1 | Benign | N = 28 | 0.93 | 0.95 |
| | vs. | 1-Methoxymethyl-2- | (90%/73%) | (90%/82%) |
| | VLR/LR/FIR/ | phenylthioindole-3- | | |
| | UIR/HR/VHR | carbaldehyde | | |
| | PC and mPC | 2,3-Dihydroxybutanoic acid | | |
| | | 2-Hydroxypyridine | | |
| | | 2-Stearoylglycerol | | |
| | | 3-Hydroxyphenylacetic acid | | |
| | | 3-Indoleacetic acid | | |
| | | 3-Methyl-L-histidine | | |
| | | 4-Acetamidobutyric acid | | |
| | | 4-hydroxymandelic acid | | |
| | | 6-ethoxyiminohexane- | | |
| | | 1,2,3,4,5-pentol | | |
| | | alpha-Hydroxyisobutyric acid | | |
| | | D-Altrose | | |
| | | D-Gluconic acid | | |
| | | D-Lyxose | | |
| | | Galacturonic acid | | |
| | | Galangin | | |
| | | Glyceric acid | | |
| | | Lactose | | |
| | | L-Fucose | | |
| | | L-Pyroglutamic acid | | |
| | | Monopalmitin | | |
| | | Ononitol | | |
| | | Oxamide | | |
| | | Palmitic acid | | |
| | | Pseudouridine | | |
| | | Pyruvic acid | | |
| | | Ribitol | | |
| | | trans-Aconitic acid | | |
| 2 | Benign and | N = 22 | 0.87 | 0.90 |
| | VLR/LR PC | 1-Stearoyl-rac-glycerol | (90%/62%) | (90%/72%) |
| | vs. | 2,5-Dipropyltetrahydrofuran | | |
| | FIR/UIR/HR/ | 3,4-Dihydroxyphenylacetic | | |
| | VHR PC and | acid | | |
| | mPC | Acetamide | | |
| | | Beta-Alanine | | |
| | | Cyclohexylamine | | |
| | | Ethyl 1-penten-3-ynesulfonate | | |
| | | Galacturonic acid | | |
| | | Galangin | | |
| | | Glyceric acid | | |
| | | Guanidinoacetic acid | | |
| | | Levulinic acid | | |
| | | Monopalmitin | | |
| | | Ononitol | | |
| | | Palmitic acid | | |
| | | p-Tolyl-beta-D- | | |
| | | glucopyranosid-uronsaeure | | |
| | | Quinic acid | | |
| | | Stearic acid | | |
| | | Sucrose | | |
| | | Uric acid | | |
| | | Xanthine | | |
| | | Xylitol | | |
| 3 | Benign and | N = 27 | 0.83 | 0.90 |
| | VLR/LR/FIR | (22S,23S,25R)-3β-methoxy- | (90%/55%) | (90%/70%) |
| | PC | 16β,23:22,26-diepoxy-5α- | | |
| | vs. | cholestane | | |
| | UIR/HR/VHR | 1-Methoxymethyl-2- | | |
| | PC and mPC | phenylthioindole-3- | | |
| | | carbaldehyde | | |
| | | 1-Stearoyl-rac-glycerol | | |
| | | 2,4-Dihydroxybutanoic acid | | |
| | | 2,5-Dipropyltetrahydrofuran | | |
| | | 4-hydroxymandelic acid | | |
| | | Acetamide | | |
| | | Arabinofuranose | | |
| | | Beta-Alanine | | |
| | | Daidzein | | |
| | | D-Allose | | |
| | | DL-isoleucine | | |
| | | D-tagatofuranose | | |
| | | Ethanolamine | | |
| | | Galangin | | |
| | | Guanidinoacetic acid | | |
| | | L-Arabinitol | | |
| | | Levulinic acid | | |
| | | L-Lactic acid | | |
| | | Monopalmitin | | |
| | | Palmitic acid | | |
| | | Pseudouridine | | |
| | | Quinic acid | | |
| | | Stearic acid | | |
| | | Sucrose | | |
| | | Tartronic acid | | |
| | | Xanthine | | |
| 4 | Benign + | N = 25 | 0.78 | 0.82 |
| | GS < 7 PC | (4RS,5SR)-5-hydroperoxy-4- | (90%/38%) | (90%/45%) |
| | vs. | decanol | | |
| | GS ≥ 7 PC | 2,5-Dipropyltetrahydrofuran | | |
| | | 3,4,5-Trihydroxypentanoic | | |
| | | acid | | |
| | | 4-Hydroxybenzoic acid | | |
| | | 6-ethoxyiminohexane- | | |
| | | 1,2,3,4,5-pentol | | |
| | | Acetamide | | |
| | | Arabinofuranose | | |
| | | Beta-Alanine | | |
| | | D-Allose | | |
| | | DL-4-Hydroxy-3- | | |
| | | methoxymandelic acid | | |
| | | Ethyl 1-penten-3-ynesulfonate | | |
| | | Galacturonic acid | | |
| | | Galangin | | |
| | | Glyceric acid | | |
| | | Guanidinoacetic acid | | |
| | | Hippuric Acid | | |
| | | Levulinic acid | | |
| | | L-Pyroglutamic acid | | |
| | | Pantothenic acid | | |
| | | Pseudouridine | | |
| | | Pyruvic acid | | |
| | | Quinic acid | | |
| | | Tartronic acid | | |
| | | Uric acid | | |
| | | Xanthine | | |

What is claimed is:

1. A method to characterize prostate cancer in a subject in need thereof, comprising detecting a level of a prostate cancer marker in a biological sample from the subject, wherein the prostate cancer marker comprises one or more of metabolite markers in Tables 1, 2, 5 and 6.

2. The method of claim 1, wherein the prostate cancer marker comprises at least one metabolite marker selected from the group consisting of Ethanimidic acid, N-(trimethylsilyl)-, trimethylsilyl ester; ethanolamine; Glycine, di-TMS; pyruvic acid; Beta-alanine 1; L-(+) lactic acid; 2-hydroxypyridine; Diethanolamine, 3TMS derivative; glyceric acid; Pentenoic acid, 4-[(trimethylsilyl)oxy]-, trimethylsilyl ester; guanidinoacetic acid 2; tartronic acid; Butanoic acid, 2,4-bis[(trimethylsilyl)oxy]-, trimethylsilyl ester; L-pyroglutamic acid; DL-isoleucine 2; 1H-Indole, 1-(trimethylsilyl)-5-[(trimethylsilyl)oxy]; 2,3,4-Trihydroxybutyric acid tetrakis(trimethylsilyl) deriv., (, (R*,R*)—); 1-Deoxypentitol, 4TMS derivative; 4-hydroxybenzoic acid; 4-acetamidobutyric acid 1; L-glutamine 2; D-lyxose 2; Arabinofuranose, 1,2,3,5-tetrakis-O-(trimethylsilyl); xanthine; Ribitol TMS; xylitol; L-(−)-Arabitol, 5TMS derivative; Furan, tetrahydro-2,5-dipropyl-; 1,5-anhydro-D-sorbitol; L-Phenylalanine, 2TMS derivative; 3,4-Dihydroxyphenylacetic Acid, 3TMS derivative; DL-4-hydroxymandelic acid; 3-methyl-L-histidine; trans-aconitic acid; Ethyl (E)-1-penten-3-ynesulfonate; D-allose 2; D-allose 1; L-tyrosine 2; quinic acid; galacturonic acid 2; Ononitol TMS; D-Gluconic acid, 6TMS derivative; pantothenic acid 2; N-acetyl-D-mannosamine 1; D-Allose, pentakis(trimethylsilyl) ether, ethyloxime (isomer 2); Pseudo uridine penta-tms; palmitic acid; 2-phenyl-3,5,7-tris(trimethylsilyloxy)-1-benzopyran-4-one; stearic acid; Guanosine, N,N-dimethyl-1-(trimethylsilyl)-2′,3′,5′-tris-O-(trimethylsilyl)-; 1-Monopalmitin, 2TMS derivative; lactose 1; 2-Monostearin, 2TMS derivative; 1-stearoyl-rac-glycerol; and 3-Phenyl-5,10-secocholesta-1(10),2-dien-5-one.

3. The method of claim 2, wherein the biological sample is peripheral blood, sera, plasma, urine, semen, prostatic fluid, Cowper's fluid, pre-ejaculatory fluid, or any combination thereof.

4. The method of claim 3, further comprising detecting a level of prostate specific antigen in the biological sample from the subject.

5. The method of claim 4, further comprising grouping the subject by NCCN risk classification into six groups of different severities of prostate cancer, wherein the six groups are benign group, very low-risk/low-risk prostate cancer, favorable-intermediate-risk prostate cancer, unfavorable-intermediate-risk prostate cancer, high-risk/very high-risk prostate cancer, and metastasis prostate cancer group.

6. The method of claim 5, further comprising distinguishing the severity of prostate cancer in the subject in one group from the other groups.

7. The method of claim 6, further comprising distinguishing the severity of prostate cancer in the subject in more than one group from the other groups.

8. A method for determining a need of biopsy for prostate cancer diagnosis in a subject in need thereof, comprising detecting a level of a prostate cancer marker in a biological sample from the subject, wherein the prostate cancer marker is selected from the group consisting of panels in Tables 3, 4, 7 and 8.

9. The method of claim 8, wherein the prostate cancer marker is selected from the group consisting of panel 1, panel 2, panel 3, panel 4, and any combination thereof, and wherein:
panel 1 is selected from the group consisting of C₁₀H₂₁N₄O₂, C₁₂H₁₇NO, C₁₂H₂NOPS, C₁₂H₉O₉P, C₁₃H₁₉N₅O₅, C₁₇H₃₂N₃O₇, C₁₈H₁₆N₆O₃, C₁₈H₃₃NO₄, C₁₈H₄₃N₄O₃, C₁₉H₃₅NO₅, C₁₉H₃₈N₂O₃, C₂₄H₄₂N₇O₃, C₂₇H₁₂N₉, C₃₄H₂₃N₇O₅, C₅H₁ NO, C₅₁H₂₉N₅O₄, C₆H₁₅N, C₈H₉N, C₉H₄N₅O₉, C₉H₈O₂, and any combination thereof;
panel 2 is selected from the group consisting of C₁₁H₅NOPS, C₁₂H₁₆NO₇, C₁₂H₂NOPS, C₁₂H₉O₉P, C₁₃H₂₅NO₂, C₁₃H₂₅NO₃, C₁₄H₃₀N₄O₂, C₁₆H₁₃N₃O₃P, C₁₇H₄₁N₄O₃, C₁₉H₁₉N₈, C₂₂H₄₅NO₄, C₂₆H₅₁N₄O₅, C₂₆H₅₈N₁₃P, C₂₇H₁₂N₉, C₂₇H₅₅N₈O₃, C₃₀H₅₇NO₇, C₃₀H₆₄N₁₅O₂P, C₄₁H₂₃N₁₁O₂, C₅, C₅H₇NO₃, C₆HCl₅, C₆H₁₆N₃O₅, C₈H₁₆NO₅, and any combination thereof;
panel 3 is selected from the group consisting of C₁₀H₁₈N₂O₅, C₁₂H₂₁NO₄, C₁₃H₂₃NO₆, C₁₃H₂₅NO₃, C₁₄H₃₀N₄O₂, C₁₅H₃₀N₁₀OP, C₁₆H₁₃N₃O₃P, C₁₉H₃₁N₆O₂, C₂₂H₄₅NO₄, C₂₇H₁₂N₉, C₂₈H₅₇N₈O₄, C₃₀H₆₁N₈O₅, C₃₀H₆₄N₁₅O₂P, C₃₅H₇₁N₈O₇, C₄₀H₃₈N₂₂O₄, C₄₁H₂₃N₁₁O₂, C₄₃H₄₀N₂₀O₃, C₅H₁₁NO, C₅H₁₁NO₂S, C₅H₂O₂P, C₈H₁₆NO₅, and any combination thereof; and
panel 4 is selected from the group consisting of C₁₁H₂₀O₂, C₁₁H₅NOPS, C₁₂H₂NOPS, C₁₂H₂₅NO₄P, C₁₂H₉O₉P, C₁₃H₂₅NO₂, C₁₃H₂₅NO₃, C₁₄H₃₀N₄O₂, C₁₆H₃₀N₃O₂, C₁₇H₄₁N₄O₃, C₁₈H₃₄O₅, C₁₉H₃₁N₆O₂, C₂₁H₃₆N₄O₃, C₂₂H₄₅NO₄, C₂₃H₄₇N₈O₂, C₂₄H₄₁N₁₄O₈, C₂₇H₁₂N₉, C₃₀H₅₇NO₇, C₃₀H₆₄N₁₅O₂P, C₃₅H₇₁N₈O₇, C₄₃H₄₀N₂₀O₃, C₅H₁₁NO, C₅H₁₁NO₂S, C₆H₁₄N₂O₅P, and any combination thereof.

10. The method of claim 8, wherein the prostate cancer marker is selected from the group consisting of panel 5, panel 6, panel 7, panel 8, and any combination thereof, and wherein:
panel 5 is selected from the group consisting of C₁₀H₁₆O₄, C₁₀H₁₈N₂O₄, C₁₁H₂₀NO₃P₃, C₁₂H₇N₄O₂, C₁₅H₂₈N₆OP₂, C₁₆H₃₉N₈OP, C₁₉H₁₄O₃, C₂₁H₃₃N₃O₃, C₂₃H₂₇O₁₁S, C₂₃H₄₂N₇O, C₂₅H₄₆N₇O₃, C₂₇H₄₈P₂, C₂₇H₅₄O₆, C₃₃H₂₂O₇, C₃₄H₇₃N₈O₂P, C₃₈H₄₈O₁₂, C₄₀H₈₅NOP₃, C₅H₄O₃, C₅H₃N₂O₂, C₆H₁₁N₄O₃P, C₆H₁₃N₄OP₂, C7H₁₀O₄, C7H₁₇O₇P₂, C7H₆O₆S, C₈H₁₄O₄, and any combination thereof;
panel 6 is selected from the group consisting of C₁₀H₁₆O₄, C₁₁H₁₆N₄O₄, C₁₂H₇N₄O₂, C₁₄H₂₀N₂O₅, C₁₆H₃₂O₂, C₁₇H₃₄N₉O₂, C₂₁H₃₃N₃O₃, C₂₃H₂₇O₁₁S, C₂₆H₄₃NO₆, C₂₇H₄₈P₂, C₂₈H₅₂N₇O, C₃₄H₇₃N₈O₂P, C₃₈H₄₈O₁₂, C₃₉H₂₆O₇, C₄H₆O₄, C₄₀H₈₅NOP₃, C₅H₄N₄O₂, C₅H₃N₂O₂, C₆H₁₁N₄O₂P, C₆H₁₃N₄OP₂, C₆H₆N₄O₂, C₆H₈N₂O₄, C7H₁₀O₄, C7H₁₇O₇P₂, C7H₆O₆S, C₉H₁₆O₄, C₉H₉NO₃, and any combination thereof;
panel 7 is selected from the group consisting of C₁₀H₁₈N₂O₄, C₁₀H₁₉N₅P₃, C₁₂H₇N₄O₂, C₁₅H₂₈N₆O P₂, C₁₆H₃₂O₂, C₁₇H₃₄N₉O₂, C₁₉H₁₄O₃, C₂₁H₃₃N₃O₃, C₂₁H₃₉N₄OP, C₂₇H₄₈P₂, C₃₈H₄₈O₁₂, C₃₉H₂₆O₇, C₄₀H₈₅NOP₃, C₅H₁₀N₂O₃, C₅H₄N₄O₂, C₆H₁₀O₄S, C₆H₁N₄O₂P, C₆H₁₃N₄OP₂, C₆H₁₅O₈P, C7H₁₀O₄, C7H₁₇O₇P₂, C7H₂₁N₃OP₃, C7H₂₂N₄O₉PS, C7H₈O₆S, C₈H₉O₆, C₉H₁₆O₄, C₉H₁₇NO₄S, C₉H₉NO₃, and any combination thereof; and
panel 8 is selected from the group consisting of C₁₀H₁₉N₅P₃, C₁₂H₇N₄O₂, C₁₅H₂₈N₆OP₂, C₁₇H₃₄N₉O₂, C₁₇H₄₂N₅OP₂, C₂₁H₃₃N₃O₃, C₂₂H₃₈N₇O, C₂₄H₄₀N₄O₃, C₂₅H₄₆N₇O₃, C₂₅H₅₀O₆, C₂₇H₅₄O₆, C₃₉H₂₆O₇, C₃₉H₇₈O₆, C₄₀H₈₅NOP₃, C₆H₁₁N₄O₂P, C₆H₁₅O₈P, C₆H₅N₂OP, C₆H₈O₆S, C7H₁₀O₄, C7H₁₇O₇P₂, C7H₂₁N₃OP₃, C₈H₁₆N₂O₅P, C₈H₁₈NO₆P, C₈H₄N₄O₃, C₉H₁₆O₄, C₉H₉NO₃, and any combination thereof.

11. The method of claim 8, wherein the prostate cancer marker is selected from the group consisting of panel 9, panel 10, panel 11, panel 12, and any combination thereof, and wherein:
panel 9 is selected from the group consisting of Pyruvic acid, 4-Acetamidobutyric acid, 1,5-Anhydro-D-glucitol, Beta-Alanine, Glyceric acid, D-Lyxose, Galacturonic acid, D-Allose, L-Tyrosine, 3-Methyl-L-histidine, L-Glutamine, L-Pyroglutamic acid, Guanidinoacetic acid, Lactose, 2-Hydroxypyridine, N-acetyl-D-mannosamine, Palmitic acid, 1-Deoxy-d-ribitol, Monopalmitin, 2-Stearoylglycerol, Galangin, 6-ethoxyiminohexane-1,2,3,4,5-pentol, D-Gluconic acid, N,N-Dimethylguanosine, Pseudouridine, Ribitol, and any combination thereof;
panel 10 is selected from the group consisting of Pyruvic acid, Xanthine, 4-Acetamidobutyric acid, 1,5-Anhydro-D-glucitol, Beta-Alanine, 1-Stearoyl-rac-glycerol, Glyceric acid, Galacturonic acid, Quinic acid, Xylitol, L-Pyroglutamic acid, Guanidinoacetic acid, Lactose, Ononitol, 5-Hydroxyindole, Monopalmitin, Galangin, 3,4-Dihydroxyphenylacetic acid, 3-Phenyl-5,10-secocholesta-1 (10),2-dien-5-one, Acetamide, Ethyl 1-penten-3-ynesulfonate, 2,5-Dipropyltetrahydrofuran, L-Phenylalanine, Pseudouridine, and any combination thereof;
panel 11 is selected from the group consisting of L-Lactic acid, Xanthine, 4-Acetamidobutyric acid, Beta-Alanine, 1-Stearoyl-rac-glycerol, 4-hydroxymandelic acid, trans-Aconitic acid, D-Allose, Tartronic acid, Stearic acid, L-Tyrosine, Quinic acid, Ethanolamine, Guanidinoacetic acid, DL-isoleucine, Palmitic acid, Monopalmitin, Arabinofuranose, 2,4-Dihydroxybutanoic acid, Diethanolamine, Acetamide, 2,5-Dipropyltetrahydrofuran, Glycine, L-Arabinitol, Levulinic acid, Pseudouridine, and any combination thereof; and
panel 12 is selected from the group consisting of Pyruvic acid, Xanthine, 4-Hydroxybenzoic acid, Beta-Alanine, 1-Stearoyl-rac-glycerol, Galacturonic acid, D-Allose, Tartronic acid, Quinic acid, Pantothenic acid, Xylitol, Guanidinoacetic acid, 1-Deoxy-d-ribitol, Monopalmitin, Threonic acid, Galangin, Arabinofuranose, 2,4-Dihydroxybutanoic acid, D-Gluconic acid, 2,5-Dipropyltetrahydrofuran, Levulinic acid, Pseudouridine, and any combination thereof.

12. The method of claim 8, wherein the prostate cancer marker is selected from the group consisting of panel 13, panel 14, panel 15, panel 16, and any combination thereof, and wherein:
panel 13 is selected from the group consisting of 1-Methoxymethyl-2-phenylthioindole-3-carbaldehyde, 2,3-Dihydroxybutanoic acid, 2-Hydroxypyridine, 2-Stearoylglycerol, 3-Hydroxyphenylacetic acid, 3-Indoleacetic acid, 3-Methyl-L-histidine, 4-Acetamidobutyric acid, 4-hydroxymandelic acid, 6-ethoxyiminohexane-1,2,3,4,5-pentol, alpha-Hydroxyisobutyric acid, D-Altrose, D-Gluconic acid, D-Lyxose, Galacturonic acid, Galangin, Glyceric acid, Lactose, L-Fucose, L-Pyroglutamic acid, Monopalmitin, Ononitol, Oxamide, Palmitic acid, Pseudouridine, Pyruvic acid, Ribitol, trans-Aconitic acid and any combination thereof;
panel 14 is selected from the group consisting of 1-Stearoyl-rac-glycerol, 2,5-Dipropyltetrahydrofuran, 3,4-Dihydroxyphenylacetic acid, Acetamide, Beta-Alanine, Cyclohexylamine, Ethyl 1-penten-3-ynesulfonate, Galacturonic acid
Galangin, Glyceric acid, Guanidinoacetic acid, Levulinic acid, Monopalmitin, Ononitol, Palmitic acid, p-Tolyl-beta-D-glucopyranosid-uronsaeure, Quinic acid, Stearic acid, Sucrose, Uric acid, Xanthine, Xylitol, and any combination thereof;
panel 15 is selected from the group consisting of (22S,23S,25R)-3β-methoxy-16β,23:22,26-diepoxy-5α-cholestane, 1-Methoxymethyl-2-phenylthioindole-3-carbaldehyde, 1-Stearoyl-rac-glycerol, 2,4-Dihydroxybutanoic acid, 2,5-Dipropyltetrahydrofuran, 4-hydroxymandelic acid, Acetamide, Arabinofuranose, Beta-Alanine, Daidzein, D-Allose, DL-isoleucine, D-tagatofuranose, Ethanolamine, Galangin, Guanidinoacetic acid, L-Arabinitol, Levulinic acid, L-Lactic acid, Monopalmitin, Palmitic acid, Pseudouridine, Quinic acid, Stearic acid, Sucrose, Tartronic acid, Xanthine, and any combination thereof; and
panel 16 is selected from the group consisting of (4RS,5SR)-5-hydroperoxy-4-decanol, 2,5-Dipropyltetrahydrofuran, 3,4,5-Trihydroxypentanoic acid, 4-Hydroxybenzoic acid, 6-ethoxyiminohexane-1,2,3,4,5-pentol, Acetamide, Arabinofuranose, Beta-Alanine, D-Allose, DL-4-Hydroxy-3-methoxymandelic acid, Ethyl 1-penten-3-ynesulfonate, Galacturonic acid, Galangin, Glyceric acid, Guanidinoacetic acid, Hippuric Acid, Levulinic acid, L-Pyroglutamic acid, Pantothenic acid, Pseudouridine, Pyruvic acid, Quinic acid, Tartronic acid, Uric acid, Xanthine, and any combination thereof.

13. A method for monitoring a prostate cancer subject on active surveillance (AS), comprising detecting a level of a prostate cancer marker in a biological sample from the subject, wherein the prostate cancer marker is selected from the group consisting of markers in Tables 1, 2, 5 and 6.
